(19) United States
(12) Patent Application Publication
(10) Pub. No.: US 2011/0230566 A1
(43) Pub. Date: Sep. 22, 2011

(54) PERFLUOROCARBON EYE CREAM FORMULATIONS

(76) Inventors: Maria Isabel Tamargo, Philadelphia, PA (US); Aharon Grossman, Durham, NC (US); Richard Kiral, Laguna Hills, CA (US)

(21) Appl. No.: 13/051,112

(22) Filed: Mar. 18, 2011

Related U.S. Application Data
(60) Provisional application No. 61/340,605, filed on Mar. 19, 2010.
(60) Provisional application No. 61/402,790, filed on Sep. 3, 2010.

(51) Int. Cl. A61K 31/02; A61P 17/00

(52) U.S. Cl. 514/759

(57) ABSTRACT

Disclosed are perfluorocarbon compositions for cosmetic applications, in particular, for application to the periocular skin, and methods for using the same.

This application claims the benefit of U.S. Provisional Application No. 61/340,605, filed Mar. 19, 2010 and U.S. Provisional Application No. 61/402,790, filed Sep. 3, 2010, the entire content of each of which is hereby incorporated by reference herein.

Throughout this application various publications, published patent applications, and patents are referenced. The disclosures of these documents in their entireties are hereby incorporated by reference into this application in order to more fully describe the state of the art to which this invention pertains.

### BACKGROUND OF THE INVENTION

### Periocular Skin

The skin around the eyes, or periocular skin, is is among the most delicate areas of the body, and is liable to show the signs of aging, including wrinkles, fine lines and dark under-eye circles, before the rest of the face.

Periocular skin is distinct from other parts of the skin. The differences are notably that skin in this area contains less lipid in the corneum stratum, the outermost layer of the epidermis, that the corneum stratum has fewer layers, that it has higher epidermal kinetics and that it is located close to a warm and moist environment. In addition to being thinner than skin in other area of the body, periocular skin also contains fewer oil glands. These characteristics make periocular skin especially sensitive and vulnerable to damage from various sources, including environmental damages and aging.

The skin around the eyes is also a difficult area of skin to care for.

### Perfluorocarbons

Perfluorocarbons (PFCs) possess the ability to dissolve large quantities of many gases at concentrations much larger than water, saline and plasma. PFCs that are commonly used in medical research are non-toxic, biologically inert, biostatic liquids at room temperature with densities of about 1.5-2.0 g/mL and high solubilities for oxygen and carbon dioxide. Such PFCs have been found to be efficient carriers of gases, both as emulsions for intravenous use and as neat liquids for liquid ventilation applications.

### SUMMARY OF THE INVENTION

The subject application provides for a method of delivering oxygen to a periocular skin of a subject comprising topically administering to a facial area consisting of the periocular skin of the subject a composition comprising a perfluorocarbon effective to deliver oxygen to the periocular skin.

The subject application also provides for a method of improving the appearance of a periocular skin of a subject comprising topically administering to a facial area consisting of the periocular skin of the subject a composition comprising a perfluorocarbon effective to improve the appearance of the periocular skin.

The subject application also provides for a perfluorocarbon cream composition comprising 1) a perfluorocarbon, 2) ascorbyl glucoside, 3) a first mixture comprising butylene glycol, water, niacinamide, fraxinus excelsior bark extract, silanetriol, and potassium citrate, 4) a second mixture comprising water, glycerin, steareth-20, N-hydroxysuccinimide, chrysin, palmitoyl oligopeptide and palmitoyl tetrapeptide-7 and 5) a third mixture comprising glycerin, water, butylene glycol, carbomer, polysorbate 20, palmitoyl oligopeptide, and palmitoyl tetrapeptide-7.

The subject application also provides for a method of decreasing the Fitzpatrick Wrinkle Assessment Scale score of a subject's skin comprising topically administering to the skin of the subject a composition comprising a perfluorocarbon effective to decrease the Fitzpatrick Wrinkle Assessment Scale score.

The subject application also provides for a method of improving the Global Aesthetic Improvement Scale score of a subject's skin comprising topically administering to the skin of the subject a composition comprising a perfluorocarbon effective to increase the Global Aesthetic Improvement Scale score.

### BRIEF DESCRIPTION OF THE FIGURES

FIG. 1: shows percentage of subjects exhibiting change in FWAS score in Example 14. Greater than 80.00% of subjects exhibited an improvement in facial wrinkles and elastosis after 8 weeks.

FIG. 2: shows subject GAIS scores in Example 14. Greater than 80.00% of subjects believe their facial appearance has improved after 8 weeks.

FIG. 3: shows Investigator GAIS Scores in Example 14. Greater than 90.00% of subjects were graded as improved by an investigator after 8 weeks.

FIG. 4: shows Plot of Means and Confidence Intervals (95.00%)—Breadth Parameter in Example 14. Subjects experienced a significant decrease in the breadth of fine lines and a non-significant trend in reduction in the breadth of major lines, resulting in smoother appearing skin.

### DETAILED DESCRIPTION OF THE INVENTION

### Embodiments of the Invention

The subject application provides for a method of delivering oxygen to a periocular skin of a subject comprising topically administering to a facial area consisting of the periocular skin of the subject a composition comprising a perfluorocarbon effective to deliver oxygen to the periocular skin.

The subject application also provides for a method of improving the appearance of a periocular skin of a subject comprising topically administering to a facial area consisting of the periocular skin of the subject a composition comprising a perfluorocarbon effective to improve the appearance of the periocular skin.

In one embodiment, the molecular formula of the perfluorocarbon consists fluorine atoms and 9-12 carbon atoms. In another embodiment, the perfluorocarbon is perfluoro(tert-butylcyclohexane). In another embodiment, the composition is in the form of a gel. In yet another embodiment, the composition is in the form of a cream.

In one embodiment, the composition is administered periodically. In another embodiment, the composition is administered twice daily. In another embodiment, the administration is for a period of greater than 3 weeks. In yet another embodiment, the administration is for a period of 8 weeks or more.

In one embodiment, the subject's Fitzpatrick Wrinkle Assessment Scale score is decreased. In another embodiment, the subject's Fitzpatrick Wrinkle Assessment Scale score is decreased by at least 1 point. In another embodiment, the subject's Fitzpatrick Wrinkle Assessment Scale score is decreased by at least 2 points. In yet another embodiment, the subject's Global Aesthetic Improvement Scale score is improved.

In one embodiment, the improvement in appearance is the reduction of the severity of fine lines, wrinkles, skin elastosis, puffiness, dark circles, under-eye circles, bags and/or dark blemishes.

The subject application also provides for a perfluorocarbon cream composition comprising 1) a perfluorocarbon, 2) ascorbyl glucoside, 3) a first mixture comprising butylene glycol, water, niacinamide, fraxinus excelsior bark extract, silanetriol, and potassium citrate, 4) a second mixture comprising water, glycerin, steareth-20, N-hydroxysuccinimide, chrysin, palmitoyl oligopeptide and palmitoyl tetrapeptide-7 and 5) a third mixture comprising glycerin, water, butylene glycol, carbomer, polysorbate 20, palmitoyl oligopeptide, and palmitoyl tetrapeptide-7.

In one embodiment, the molecular formula of the perfluorocarbon consists fluorine atoms and 9-12 carbon atoms. In another embodiment, the perfluorocarbon is perfluoro(tert-butylcyclohexane).

In one embodiment, the perfluorocarbon is 1-90 wt % relative to the total weight of the composition. In another embodiment, the perfluorocarbon is 5-90 wt % relative to the total weight of the composition. In another embodiment, the perfluorocarbon is 15-90 wt % relative to the total weight of the composition. In yet another embodiment, the perfluorocarbon is 17-25 wt % relative to the total weight of the composition.

In one embodiment, the ascorbyl blucoside is 1-10 wt % relative to the total weight of the composition. In another embodiment, the first mixture is 1-10 wt % relative to the total weight of the composition. In another embodiment, the second mixture is 1-10 wt % relative to the total weight of the composition. In another embodiment, the third mixture is 1-10 wt % relative to the total weight of the composition.

In one embodiment, the perfluorocarbon cream composition further comprises a pharmaceutically acceptable carriers or a cosmetic carrier.

In one embodiment, the perfluorocarbon cream composition is characterized by it having a viscosity of 5,000-30,000 cps at 25° C. In another embodiment, the perfluorocarbon cream composition is characterized by it having a viscosity of 10,000-20,000 cps at 25° C.

In one embodiment, the perfluorocarbon cream composition is characterized by it having a specific gravity of 1.01-1.82. In another embodiment, the perfluorocarbon cream composition is characterized by it having a specific gravity of 1.14-1.18.

The subject application also provides for a method of decreasing the Fitzpatrick Wrinkle Assessment Scale score of a subject's skin comprising topically administering to the skin of the subject a composition comprising a perfluorocarbon effective to decrease the Fitzpatrick Wrinkle Assessment Scale score.

The subject application also provides for a method of improving the Global Aesthetic Improvement Scale score of a subject's skin comprising topically administering to the skin of the subject a composition comprising a perfluorocarbon effective to increase the Global Aesthetic Improvement Scale score.

All combinations of the various elements described herein are within the scope of the invention.

Terms

As used herein, and unless stated otherwise, each of the following terms shall have the definition set forth below.

“Administering to the subject” means the giving of, dispensing of, or application of medicines, drugs, or remedies to a subject to relieve, cure, or reduce the symptoms associated with a condition, e.g., a pathological condition. “Topical administration” of a composition as used herein shall mean application of the composition to the skin of a subject. In an embodiment, topical administration of a composition is application of the composition to the epidermis of a subject.

“Biologically active agent” means a substance which has a beneficial effect on living tissue.

“Cream” means a liquid or semi-liquid colloid at ambient temperature wherein the dispersed phase is dispersed in a liquid/semi-liquid continuous medium. The cream is more viscous than a liquid but less viscous than a gel. The use of the term “cream” in this application specifically excludes “gel”.

“Effective” as in an amount effective to achieve an end means the quantity of a component that is sufficient to yield a desired therapeutic response with a reasonable benefit/risk ratio of side effects. For example, an amount effective to deliver oxygen to a subject's periocular skin, or an amount effective to improve the overall appearance of a subject's periocular skin, without causing unreasonable adverse side effects. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

“Fitzpatrick Wrinkle Assessment Scale” or “FWAS” is a 9-grade scale for assessing the diverse aspects of aging skin. FWAS ranks the depth of the wrinkle (e.g., fine lines or deep wrinkles) and elastosis, the process of increasing the amount of elastic tissue and improving the pliability of the skin. FWAS is commonly used in dermatology to determine the effectiveness of skin care treatments and therapies.

“Gel” means a semi-solid or solid colloid (depending on concentration and/or temperature) of a solid/semi-solid and a liquid wherein a liquid dispersed phase is dispersed in a solid/semi-solid continuous medium. Some gels become fluids due to agitation then resume their gel structure when allowed to be undisturbed. Common pharmaceutical gels are solids which when applied and with motion allow the product to become temporarily a liquid phase so it applies smoothly, then becomes tacky then dries. Other gels are semi solid which are a semi-liquid, semi-solid mixture & when applied become tacky then dry.

“Global Aesthetic Improvement Scale” or “GAIS” is another commonly used scale used for assessing changes to skin after treatment is applied. The GAIS rates changes on a scale of one-to-five (1-5), with one (1) being the most improved and five (5) indicating that the appearance has worsened.

“Oxygenated perfluorocarbon” is a perfluorocarbon which is carrying oxygen at, for example, saturation or sub-saturation levels.

“Periocular skin” means the skin in the region around the eye, specifically, the skin in the region bounded by the brow superiorly, the infraorbital rim inferiorly, the nose medially and the lateral orbital rim.

“Pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject. The carrier may be liquid or solid and is selected with the planned manner of administration in mind.

“wt %” when referring to the percentage of a component in the claimed cream composition is percentage of the weight of the component in the cream relative to the total weight of the cream.

Perfluoro(tert-butylcyclohexane)

PFCs include perfluoro(tert-butylcyclohexane) (C₁₀F₂₀, CAS No. 84808-64-0) which is available, for example, as Oxycyte® from Oxygen Biotherapeutics Inc., Costa Mesa, Calif. In an embodiment, the perfluoro(tert-butylcyclohexane) has the following structure:

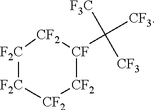

Physical properties of perfluoro(tert-butylcyclohexane) are as follows:

| Molecular Formula | C₁₀F₂₀ |
|---|---|
| Molecular Weight (g/mol) | 500.08 |
| Physical State @ Room Temp. | Liquid |
| Density (g/mL) | 1.97 |
| Boiling Point (° C.) | 147 |
| Vapor Pressure (mmHg) @ 25° C. | 3.8 |
| Vapor Pressure (mmHg) @ 37° C. | 4.4 |
| Kinematic Viscosity (cP) | 5.378 |
| Refractive Index @ 20° C. | 1.3098 |
| Calculated Dipole Moment (Debye) | 0.287 |
| Calculated Surface Tension (dyne/cm) | 14.4 |

Perfluoro(tert-butylcyclohexane) can carry about 43 mL of oxygen per 100 mL of PFC, and 196 mL of CO₂ per 100 mL of PFC at body temperature.

Oxycyte® is a perfluorocarbon emulsion oxygen carrier. The active ingredient in Oxycyte®, perfluoro(tert-butylcyclohexane) (C₁₀F₂₀, MW˜500), also known as F-tert-butylcyclohexane or “FtBu”, is a saturated alicyclic PFC. Perfluoro(tert-butylcyclohexane) is a colorless, completely inert, non-water soluble, non-lipophilic molecule, which is twice as dense as water, and boils at 147° C. Oxycyte® can be used in the PFC compositions, methods and uses described herein.

Being that the PFCs are slightly lipophilic at body temperature and would help in the transport of oxygen into and removal of carbon dioxide from, e.g., periocular skin. Perfluoro(tert-butylcyclohexane) is only slightly lipophilic at body temperature and not lipophilic at room temperature.

The Perfluorocarbon Cream

In one embodiment of the present invention the perfluorocarbon composition is formulated as a cream. The perfluorocarbon cream provided by this application can contain components from the following list: perfluorocarbon, water, cyclopentasiloxane, propanediol, caprylic/capric triglyceride, butylene glycol, glycerin, Butyrospermum parkii (shea butter) dimethicone, cetyl phosphate, stearic acid, Limnanthes alba (meadowfoam) seed oil, glyceryl stearate, PEG-100 sterate, ascorbyl glucoside, Helianthus annuus (sunflower) seed oil unsaponifiables, Persea gratissima (avocado) oil unsaponifiables, Fraxinus excelsior bark extract, Avena sativa (oat) kemel extract, dipotassium glycyrrhizate, niacinamide, palmitoyl oligopeptide, palmitoyl tetrapeptide-7, acrylates/dimethicone copolymer, steareth-20, silanetriol, N-hydroxysuccinimide, chrysin, polyurethane-40, silica, potassium citrate, polysorbate 20, carbomer, disodium EDTA, sodium hydroxide, caprylyl glycol, chlorphenesin, phenoxyethanol, fragrance (parfum) and Green 5.

In one particular embodiment of the present invention, the cream is formulated as follows:

TABLE 1
Representative Composition of Perfluorocarbon Cream.

| CAS # | Trade Name | INCI Name | Wt. % | Breakdown |
|---|---|---|---|---|
| N/A | Perfluoro-tert- | Perfluoro-tert- | 15.00-90.00% | 97% |
| | butylclyclohexane | butylclyclohexane | | |
| 7732-18-5 | Deionized Water | Water (Aqua) | 15.00-90.00% | 100% |
| 76050-42-5 | Carbopol Ultrez-10 | Carbomer | 0-1.00% | 100% |
| 504-63-2 | Zemea | Propanediol | 1-10.00% | 100% |
| 139-33-3 | Dissolvine Na-2-P | Disodium EDTA | 0-1.00% | 100% |
| 91770-40-0 | Avocadin | Persea gratissima | 0-1.00% | 100% |
| | | (Avocado) Oil | | |
| | | Unsaponifiables | | |
| 68920-03-6 | Shea Butter | Butyrospermum parkii | 1-10.00% | 100% |
| | “Ultra Refined” | (Shea Butter) | | |
| 57-11-4 | Stearic Acid | Stearic Acid | 1-10.00% | 100% |
| 3539-43-3 | Amphisol A | Cetyl Phosphate | 1-10.00% | 100% |
| 31566-31-1 | Simulsol 165 | Glyceryl Stearate | 1-10.00% | 50% |
| 9004-99-3 | | PEG-100 Stearate | | 50% |
| 63148-62-9 | Dow Corning 200 | Dimethicone | 1-10.00% | 100% |
| | Fluid 350 CST | | | |
| 541-02-6 | Volasil 995 | Cyclopentasiloxane | 1-10.00% | 99% |
| 65381-09-1 | Myritol 312 | Caprylic/Capric | 1-10.00% | 100% |
| | | Triglyceride | | |
| 541-02-6 | KP-545 | Cyclopentasiloxane | 1-10.00% | 70% |
| N/A | | Acrylates/Dimethicone | | 30% |
| | | Copolymer | | |
| 8001-21-6 | Soline | Helianthus annuus | 0-1.00% | 100% |
| | | (Sunflower) Seed Oil | | |
| | | Unsaponifiables | | |
| N/A | Botanol MO | Limnanthes alba | 1-10.00% | 100% |
| | | (Meadowfoam) Seed Oil | | |
| 1310-73-2 | Sodium Hydroxide, | Sodium Hydroxide | 0-1.00% | 100% |
| | Pellets, NF | | | |
| 122-99-6 | Mikrokill Cos | Phenoxyethanol | 1-10.00% | 66% |
| 1117-86-8 | | Caprylyl Glycol | | 15% |
| 104-29-0 | | Chlorphenesin | | 19% |
| 68797-35-3 | ARG-DPG | Dipotassium | 0-1.00% | 100% |
| | | Glycyrrhizate | | |
| 129499-78-1 | AA2G | Ascorbyl Glucoside | 1-10.00% | 100% |
| 7732-18-5 | Drago-Calm 674463 | Water (Aqua) | 0-1.00% | 49.5% |
| 56-81-5 | | Glycerin | | 49.5% |
| 84012-26-0 | | Avena sativa (Oat) | | 1% |
| | | Kernel Extract | | |
| 107-88-0 | Cytobiol Lumin-Eye | Butylene Glycol | 1-10.00% | 37% |
| 7732-18-5 | | Water (Aqua) | | 37% |
| 98-92-0 | | Niacinamide | | 18% |
| 84625-28-5 | | Fraxinus excelsior | | 6.2% |
| | | Bark Extract | | |
| N/A | | Silanetriol | | 0.4% |
| 866-84-2 | | Potassium Citrate | | 0.4% |
| 7732-18-5 | Haloxyl | Water (Aqua) | 1-10.00% | 80.7750% |
| 56-81-5 | | Glycerin | | 15% |
| 9005-00-9 | | Steareth-20 | | 4% |
| 6066-82-6 | | N-Hydroxysuccinimide | | 0.2% |
| 480-40-0 | | Chrysin | | 0.01% |
| 147732-56-7 | | Palmitoyl Oligopeptide | | 0.01% |
| 221227-05-0 | | Palmitoyl Tetrapeptide-7 | | 0.005% |
| 56-81-5 | Matrixyl 3000 | Glycerin | 1-10.00% | 53.4850% |
| 7732-18-5 | | Water (Aqua) | | 25% |
| 107-88-0 | | Butylene Glycol | | 20% |
| 76050-42-5 | | Carbomer | | 1% |
| 9005-64-5 | | Polysorbate 20 | | 0.5% |
| 147732-56-7 | | Palmitoyl Oligopeptide | | 0.01% |
| 221227-05-0 | | Palmitoyl Tetrapeptide-7 | | 0.005% |
| N/A | Fragrance - “Silky Skin” - | Fragrance (Parfum) | 0-1.00% | 100% |
| | #6110985 | | | |
| 112945-52-5 | ChronoSphere Opticals | Silica | 1-10.00% | 3% |
| N/A | Brite | Polyurethane-40 | | 5% |
| 4403-90-1 | | Green 5 | | 92% |

The PFC composition disclosed herein can be used as a vehicle to deliver oxygen to periocular skin tissue. The PFC composition disclosed herein can increase the oxygen concentration in the treated skin locally as compared to the untreated skin. To further increase oxygen concentration, the PFC composition can be pre-loaded with molecular oxygen. The composition can deliver oxygen to the tissue via a diffusion gradient.

The perfluorocarbon employed in the compositions and methods described herein may be in compositions which further comprise pharmaceutically acceptable carrier or cosmetic carrier and adjuvant(s) suitable for topical administration. Compositions suitable for topical administration are well known in the pharmaceutical and cosmetic arts. These compositions can be adapted to comprise the oxygenated perfluorocarbon. The composition employed in the methods described herein may also comprise a pharmaceutically acceptable additive.

The multiplicity of configurations may contain additional beneficial biologically active agents which further promote tissue health.

The compositions of this invention may be administered in forms detailed herein. The use of perfluorocarbon may be a component of a combination therapy or an adjunct therapy. The combination therapy can be sequential or simultaneous. The compounds and compositions can be administered independently by the same route or by two or more different routes of administration depending on the dosage forms employed.

The dosage of the compounds and compositions administered in treatment will vary depending upon factors such as the pharmacodynamic characteristics of a specific therapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds and compositions may comprise a single compound or mixtures thereof with other compounds. The compounds can be introduced directly into the targeted tissue, using dosage forms well known to those of ordinary skill in the cosmetic and pharmaceutical arts.

The compounds and compositions can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical and cosmetic practices. The compounds can be administered alone but are generally mixed with a pharmaceutically acceptable carrier. This carrier can be a solid or liquid, and the type of carrier is generally chosen based on the type of administration being used. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences, Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). All of the aforementioned publications are incorporated by reference herein.

The PFC compositions may contain the any of the following non-toxic auxiliary substances:

The PFC compositions may contain antibacterial agents which are non-injurious in use, for example, thimerosal, benzalkonium chloride, methyl and propyl paraben, benzyldodecinium bromide, benzyl alcohol, or phenylethanol.

The PFC compositions may also contain buffering ingredients such as sodium acetate, gluconate buffers, phosphates, bicarbonate, citrate, borate, ACES, BES, BICINE, BIS-Tris, BIS-Tris Propane, HEPES, HEPPS, irnidazole, MES, MOPS, PIPES, TAPS, TES, and Tricine.

The PFC compositions may also contain a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, polyvinylpyrrolidone, isopropyl myristate and other conventionally employed acceptable carriers.

The PFC compositions may also contain non-toxic emulsifying, preserving, wetting agents, bodying agents, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic.

The PFC compositions may also contain surfactants that might be employed include polysorbate surfactants, polyoxyethylene surfactants, phosphonates, saponins and polyethoxylated castor oils, but preferably the polyethoxylated castor oils. These surfactants are commercially available. The polyethoxylated castor oils are sold, for example, by BASF under the trademark Cremaphor.

The PFC compositions may also contain wetting agents commonly used in ophthalmic solutions such as carboxymethylcellulose, hydroxypropyl methylcellulose, glycerin, mannitol, polyvinyl alcohol or hydroxyethylcellulose and the diluting agent may be water, distilled water, sterile water, or artificial tears, wherein the wetting agent is present in an amount of about 0.001% to about 10%.

The formulation of this invention may be varied to include acids and bases to adjust the pH; tonicity imparting agents such as sorbitol, glycerin and dextrose; other viscosity imparting agents such as sodium carboxymethylcellulose, microcrystalline cellulose, polyvinylpyrrolidone, polyvinyl alcohol and other gums; suitable absorption enhancers, such as surfactants, bile acids; stabilizing agents such as antioxidants, like bisulfites and ascorbates; metal chelating agents, such as sodium edetate; and drug solubility enhancers, such as polyethylene glycols. These additional ingredients help make commercial solutions with adequate stability so that they need not be compounded on demand.

Other materials as well as processing techniques and the like are set forth in Part 8 of Remington's Pharmaceutical Sciences, 17th edition, 1985, Mack Publishing Company, Easton, Pa., and International Programme on Chemical Safety (IPCS), which is incorporated herein by reference.

All combinations of the various elements are within the scope of the invention.

It is understood that where a parameter range is provided, all integers within that range, and tenths thereof, are also provided by the invention. For example, “20-30 wt %” includes 20.0 wt %, 20.1 wt %, 20.2 wt %, 20.3 wt %, 20.4 wt % etc. up to 30.0 wt %.

Cosmetic Use for the Periocular Skin

The PFC compositions described herein (e.g., a perfluorocarbon cream) can be used as a cosmetic agent to improve the overall appearance of the skin and promote anti-aging, especially in the periocular skin. The PFC composition can be used for reducing skin imperfections such as fine lines, wrinkles, puffiness, dark (under-eye) circles, bags or dark blemishes around the eye. The PFC composition can also be used for the promotion of skin firmness.

Oxygen levels in the skin decrease with age, making the appearance of fine lines and wrinkles more noticeable. A lack of oxygen at the cellular level can cause skin to age prematurely, increasing the appearance of fine lines and age spots, making skin look dry and dull. Applying an oxygen-rich perfluorocarbon composition (e.g., a perfluorocarbon cream) to the skin can enhance oxygen levels in the skin, promote cell turnover and repair, reduce and/or prevent fine lines and wrinkles, thus improving overall appearance and feel of the skin.

In addition, oxygen can inhibit the destructive enzyme collagenase which breaks down collagen. Collagen is one of the structural substances that supports the skin's surface. By supporting collagen production (by inhibiting collagenase through higher oxygen levels), the skin can be firmer and look more youthful.

Therefore, the PFC composition can diminish fine lines and wrinkles by using oxygen to activate the skin regenerative functions. Moreover, the PFC composition can increase the firmness and elasticity of the skin by activating collagen and elastin creation.

The PFC composition can be a component of a combination therapy/treatment or an adjunct therapy/treatment. For example, the PFC cream can be administered in combination with another agent, e.g., a moisturizer, to improve skin appearance and/or improve skin health.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Testing for Oxycyte® Toxicity

An Oxycyte® emulsion (60% wt/vol. PFC) was tested systemically via intravenous administration in Sprauge Dawley rats, Cynomolgus Monkeys and humans.

The Oxycyte® emulsion was found to be well tolerated and had no toxicity.

### Example 2

### Representative Composition of the PFC Cream

TABLE 2
First Representative Composition

| CAS # | Trade Name | INCI Name | Wt. % |
|---|---|---|---|
| N/A | Perfluoro-tert- | Perfluoro-tert- | 25.50% |
| | butylclyclohexane | butylclyclohexane | |
| 7732-18-5 | Deionized Water | Water (Aqua) | 35.50% |
| 76050-42-5 | Carbopol Ultrez-10 | Carbomer | 0.35% |
| 504-63-2 | Zemea | Propanediol | 5.50% |
| 139-33-3 | Dissolvine Na-2-P | Disodium EDTA | 0.15% |
| 91770-40-0 | Avocadin | Persea gratissima | 0.65% |
| | | (Avocado) Oil | |
| | | Unsaponifiables | |
| 68920-03-6 | Shea Butter | Butyrospermum parkii | 3.80% |
| | “Ultra Refined” | (Shea Butter) | |
| 57-11-4 | Stearic Acid | Stearic Acid | 1.10% |
| 3539-43-3 | Amphisol A | Cetyl Phosphate | 1.50% |
| 31566-31-1 | Simulsol 165 | Glyceryl Stearate | 2.50% |
| 9004-99-3 | | PEG-100 Stearate | |
| 63148-62-9 | Dow Corning 200 | Dimethicone | 2.40% |
| | Fluid 350 CST | | |
| 541-02-6 | Volasil 995 | Cyclopentasiloxane | 2.50% |
| 65381-09-1 | Myritol 312 | Caprylic/Capric | 1.80% |
| | | Triglyceride | |
| 541-02-6 | KP-545 | Cyclopentasiloxane | 2.50% |
| N/A | | Acrylates/Dimethicone | |
| | | Copolymer | |
| 8001-21-6 | Soline | Helianthus annuus | 0.35% |
| | | (Sunflower) Seed Oil | |
| | | Unsaponifiables | |
| N/A | Botanol MO | Limnanthes alba | 1.80% |
| | | (Meadowfoam) Seed Oil | |
| 1310-73-2 | Sodium Hydroxide, | Sodium Hydroxide | 0.37% |
| | Pellets, NF | | |
| 122-99-6 | Mikrokill Cos | Phenoxyethanol | 1.40% |
| 1117-86-8 | | Caprylyl Glycol | |
| 104-29-0 | | Chlorphenesin | |
| 68797-35-3 | ARG-DPG | Dipotassium | 0.09% |
| | | Glycyrrhizate | |
| 129499-78-1 | AA2G | Ascorbyl Glucoside | 2.10% |
| 7732-18-5 | Drago-Calm | Water (Aqua) | 0.12% |
| 56-81-5 | 674463 | Glycerin | |
| 84012-26-0 | | Avena sativa (Oat) | |
| | | Kernel Extract | |
| 107-88-0 | Cytobiol | Butylene Glycol | 3.20% |
| 7732-18-5 | Lumin-Eye | Water (Aqua) | |
| 98-92-0 | | Niacinamide | |
| 84625-28-5 | | Fraxinus excelsior | |
| | | Bark Extract | |
| N/A | | Silanetriol | |
| 866-84-2 | | Potassium Citrate | |
| 7732-18-5 | Haloxyl | Water (Aqua) | 1.80% |
| 56-81-5 | | Glycerin | |
| 9005-00-9 | | Steareth-20 | |
| 6066-82-6 | | N-Hydroxysuccinimide | |
| 480-40-0 | | Chrysin | |
| 147732-56-7 | | Palmitoyl Oligopeptide | |
| 221227-05-0 | | Palmitoyl Tetrapeptide-7 | |
| 56-81-5 | Matrixyl 3000 | Glycerin | 1.50% |
| 7732-18-5 | | Water (Aqua) | |
| 107-88-0 | | Butylene Glycol | |
| 76050-42-5 | | Carbomer | |
| 9005-64-5 | | Polysorbate 20 | |
| 147732-56-7 | | Palmitoyl Oligopeptide | |
| 221227-05-0 | | Palmitoyl Tetrapeptide-7 | |
| N/A | Fragrance - | Fragrance (Parfum) | 0.02% |
| | “Silky Skin” - | | |
| | #6110985 | | |
| 112945-52-5 | ChronoSphere | Silica | 1.50% |
| N/A | Opticals Brite | Polyurethane-40 | |
| 4403-90-1 | | Green 5 | |

### Example 3

### Representative Composition of the PFC Cream

TABLE 3
Second Representative Composition

| CAS # | Trade Name | INCI Name | Wt. % |
|---|---|---|---|
| N/A | Perfluoro-tert- | Perfluoro-tert- | 40.00% |
| | butylclyclohexane | butylclyclohexane | |
| 7732-18-5 | Deionized Water | Water (Aqua) | 20.00% |
| 76050-42-5 | Carbopol Ultrez-10 | Carbomer | 0.60% |
| 504-63-2 | Zemea | Propanediol | 5.03% |
| 139-33-3 | Dissolvine Na-2-P | Disodium EDTA | 0.02% |
| 91770-40-0 | Avocadin | Persea gratissima | 0.80% |
| | | (Avocado) Oil | |
| | | Unsaponifiables | |
| 68920-03-6 | Shea Butter | Butyrospermum parkii | 2.50% |
| | “Ultra Refined” | (Shea Butter) | |
| 57-11-4 | Stearic Acid | Stearic Acid | 1.80% |
| 3539-43-3 | Amphisol A | Cetyl Phosphate | 1.70% |
| 31566-31-1 | Simulsol 165 | Glyceryl Stearate | 3.50% |
| 9004-99-3 | | PEG-100 Stearate | |
| 63148-62-9 | Dow Corning 200 | Dimethicone | 1.70% |
| | Fluid 350 CST | | |
| 541-02-6 | Volasil 995 | Cyclopentasiloxane | 3.50% |
| 65381-09-1 | Myritol 312 | Caprylic/Capric | 1.50% |
| | | Triglyceride | |
| 541-02-6 | KP-545 | Cyclopentasiloxane | 1.50% |
| N/A | | Acrylates/Dimethicone | |
| | | Copolymer | |
| 8001-21-6 | Soline | Helianthus annuus | 0.34% |
| | | (Sunflower) Seed Oil | |
| | | Unsaponifiables | |
| N/A | Botanol MO | Limnanthes alba | 1.30% |
| | | (Meadowfoam) Seed Oil | |
| 1310-73-2 | Sodium Hydroxide, | Sodium Hydroxide | 0.88% |
| | Pellets, NF | | |
| 122-99-6 | Mikrokill Cos | Phenoxyethanol | 1.20% |
| 1117-86-8 | | Caprylyl Glycol | |
| 104-29-0 | | Chlorphenesin | |
| 68797-35-3 | ARG-DPG | Dipotassium | 0.05% |
| | | Glycyrrhizate | |
| 129499-78-1 | AA2G | Ascorbyl Glucoside | 1.25% |
| 7732-18-5 | Drago-Calm | Water (Aqua) | 0.15% |
| 56-81-5 | 674463 | Glycerin | |
| 84012-26-0 | | Avena sativa (Oat) | |
| | | Kernel Extract | |
| 107-88-0 | Cytobiol | Butylene Glycol | 5.00% |
| 7732-18-5 | Lumin-Eye | Water (Aqua) | |
| 98-92-0 | | Niacinamide | |
| 84625-28-5 | | Fraxinus excelsior | |
| | | Bark Extract | |
| N/A | | Silanetriol | |
| 866-84-2 | | Potassium Citrate | |
| 7732-18-5 | Haloxyl | Water (Aqua) | 1.25% |
| 56-81-5 | | Glycerin | |
| 9005-00-9 | | Steareth-20 | |
| 6066-82-6 | | N-Hydroxysuccinimide | |
| 480-40-0 | | Chrysin | |
| 147732-56-7 | | Palmitoyl Oligopeptide | |
| 221227-05-0 | | Palmitoyl Tetrapeptide-7 | |
| 56-81-5 | Matrixyl 3000 | Glycerin | 1.60% |
| 7732-18-5 | | Water (Aqua) | |
| 107-88-0 | | Butylene Glycol | |
| 76050-42-5 | | Carbomer | |
| 9005-64-5 | | Polysorbate 20 | |
| 147732-56-7 | | Palmitoyl Oligopeptide | |
| 221227-05-0 | | Palmitoyl Tetrapeptide-7 | |
| N/A | Fragrance - | Fragrance (Parfum) | 0.08% |
| | “Silky Skin” - | | |
| | #6110985 | | |
| 112945-52-5 | ChronoSphere | Silica | 2.75% |
| N/A | Opticals Brite | Polyurethane-40 | |
| 4403-90-1 | | Green 5 | |

### Example 4

### Representative Composition of the PFC Cream

TABLE 4
Third Representative Composition

| CAS # | Trade Name | INCI Name | Wt. % |
|---|---|---|---|
| N/A | Perfluoro-tert- | Perfluoro-tert- | 46.70% |
| | butylclyclohexane | butylclyclohexane | |
| 7732-18-5 | Deionized Water | Water (Aqua) | 20.50% |
| 76050-42-5 | Carbopol Ultrez-10 | Carbomer | 0.55% |
| 504-63-2 | Zemea | Propanediol | 4.50% |
| 139-33-3 | Dissolvine Na-2-P | Disodium EDTA | 0.14% |
| 91770-40-0 | Avocadin | Persea gratissima | 0.70% |
| | | (Avocado) Oil | |
| | | Unsaponifiables | |
| 68920-03-6 | Shea Butter | Butyrospermum parkii | 2.00% |
| | “Ultra Refined” | (Shea Butter) | |
| 57-11-4 | Stearic Acid | Stearic Acid | 1.50% |
| 3539-43-3 | Amphisol A | Cetyl Phosphate | 2.10% |
| 31566-31-1 | Simulsol 165 | Glyceryl Stearate | 1.75% |
| 9004-99-3 | | PEG-100 Stearate | |
| 63148-62-9 | Dow Corning 200 | Dimethicone | 1.35% |
| | Fluid 350 CST | | |
| 541-02-6 | Volasil 995 | Cyclopentasiloxane | 1.50% |
| 65381-09-1 | Myritol 312 | Caprylic/Capric | 1.45% |
| | | Triglyceride | |
| 541-02-6 | KP-545 | Cyclopentasiloxane | 1.80% |
| N/A | | Acrylates/Dimethicone | |
| | | Copolymer | |
| 8001-21-6 | Soline | Helianthus annuus | 0.33% |
| | | (Sunflower) Seed Oil | |
| | | Unsaponifiables | |
| N/A | Botanol MO | Limnanthes alba | 1.70% |
| | | (Meadowfoam) Seed Oil | |
| 1310-73-2 | Sodium Hydroxide, | Sodium Hydroxide | 0.76% |
| | Pellets, NF | | |
| 122-99-6 | Mikrokill Cos | Phenoxyethanol | 1.10% |
| 1117-86-8 | | Caprylyl Glycol | |
| 104-29-0 | | Chlorphenesin | |
| 68797-35-3 | ARG-DPG | Dipotassium | 0.08% |
| | | Glycyrrhizate | |
| 129499-78-1 | AA2G | Ascorbyl Glucoside | 1.90% |
| 7732-18-5 | Drago-Calm | Water (Aqua) | 0.35% |
| 56-81-5 | 674463 | Glycerin | |
| 84012-26-0 | | Avena sativa (Oat) | |
| | | Kernel Extract | |
| 107-88-0 | Cytobiol | Butylene Glycol | 3.70% |
| 7732-18-5 | Lumin-Eye | Water (Aqua) | |
| 98-92-0 | | Niacinamide | |
| 84625-28-5 | | Fraxinus excelsior | |
| | | Bark Extract | |
| N/A | | Silanetriol | |
| 866-84-2 | | Potassium Citrate | |
| 7732-18-5 | Haloxyl | Water (Aqua) | 1.40% |
| 56-81-5 | | Glycerin | |
| 9005-00-9 | | Steareth-20 | |
| 6066-82-6 | | N-Hydroxysuccinimide | |
| 480-40-0 | | Chrysin | |
| 147732-56-7 | | Palmitoyl Oligopeptide | |
| 221227-05-0 | | Palmitoyl Tetrapeptide-7 | |
| 56-81-5 | Matrixyl 3000 | Glycerin | 1.00% |
| 7732-18-5 | | Water (Aqua) | |
| 107-88-0 | | Butylene Glycol | |
| 76050-42-5 | | Carbomer | |
| 9005-64-5 | | Polysorbate 20 | |
| 147732-56-7 | | Palmitoyl Oligopeptide | |
| 221227-05-0 | | Palmitoyl Tetrapeptide-7 | |
| N/A | Fragrance - | Fragrance (Parfum) | 0.04% |
| | “Silky Skin” - | | |
| | #6110985 | | |
| 112945-52-5 | ChronoSphere | Silica | 1.10% |
| N/A | Opticals Brite | Polyurethane-40 | |
| 4403-90-1 | | Green 5 | |

### Example 5

### Representative Composition of the PFC Cream

TABLE 5
Fourth Representative Composition

| CAS # | Trade Name | INCI Name | Wt. % |
|---|---|---|---|
| N/A | Perfluoro-tert- | Perfluoro-tert- | 15.00% |
| | butylclyclohexane | butylclyclohexane | |
| 7732-18-5 | Deionized Water | Water (Aqua) | 40.00% |
| 76050-42-5 | Carbopol Ultrez-10 | Carbomer | 0.30% |
| 504-63-2 | Zemea | Propanediol | 4.00% |
| 139-33-3 | Dissolvine Na-2-P | Disodium EDTA | 0.10% |
| 91770-40-0 | Avocadin | Persea gratissima | 0.10% |
| | | (Avocado) Oil | |
| | | Unsaponifiables | |
| 68920-03-6 | Shea Butter | Butyrospermum parkii | 4.00% |
| | “Ultra Refined” | (Shea Butter) | |
| 57-11-4 | Stearic Acid | Stearic Acid | 2.00% |
| 3539-43-3 | Amphisol A | Cetyl Phosphate | 2.00% |
| 31566-31-1 | Simulsol 165 | Glyceryl Stearate | 2.00% |
| 9004-99-3 | | PEG-100 Stearate | |
| 63148-62-9 | Dow Corning 200 | Dimethicone | 3.00% |
| | Fluid 350 CST | | |
| 541-02-6 | Volasil 995 | Cyclopentasiloxane | 2.70% |
| 65381-09-1 | Myritol 312 | Caprylic/Capric | 4.00% |
| | | Triglyceride | |
| 541-02-6 | KP-545 | Cyclopentasiloxane | 3.00% |
| N/A | | Acrylates/Dimethicone | |
| | | Copolymer | |
| 8001-21-6 | Soline | Helianthus annuus | 0.30% |
| | | (Sunflower) Seed Oil | |
| | | Unsaponifiables | |
| N/A | Botanol MO | Limnanthes alba | 1.50% |
| | | (Meadowfoam) Seed Oil | |
| 1310-73-2 | Sodium Hydroxide, | Sodium Hydroxide | 0.35% |
| | Pellets, NF | | |
| 122-99-6 | Mikrokill Cos | Phenoxyethanol | 1.80% |
| 1117-86-8 | | Caprylyl Glycol | |
| 104-29-0 | | Chlorphenesin | |
| 68797-35-3 | ARG-DPG | Dipotassium | 0.10% |
| | | Glycyrrhizate | |
| 129499-78-1 | AA2G | Ascorbyl Glucoside | 1.50% |
| 7732-18-5 | Drago-Calm | Water (Aqua) | 0.30% |
| 56-81-5 | 674463 | Glycerin | |
| 84012-26-0 | | Avena sativa (Oat) | |
| | | Kernel Extract | |
| 107-88-0 | Cytobiol | Butylene Glycol | 2.00% |
| 7732-18-5 | Lumin-Eye | Water (Aqua) | |
| 98-92-0 | | Niacinamide | |
| 84625-28-5 | | Fraxinus excelsior | |
| | | Bark Extract | |
| N/A | | Silanetriol | |
| 866-84-2 | | Potassium Citrate | |
| 7732-18-5 | Haloxyl | Water (Aqua) | 4.00% |
| 56-81-5 | | Glycerin | |
| 9005-00-9 | | Steareth-20 | |
| 6066-82-6 | | N-Hydroxysuccinimide | |
| 480-40-0 | | Chrysin | |
| 147732-56-7 | | Palmitoyl Oligopeptide | |
| 221227-05-0 | | Palmitoyl Tetrapeptide-7 | |
| 56-81-5 | Matrixyl 3000 | Glycerin | 4.00% |
| 7732-18-5 | | Water (Aqua) | |
| 107-88-0 | | Butylene Glycol | |
| 76050-42-5 | | Carbomer | |
| 9005-64-5 | | Polysorbate 20 | |
| 147732-56-7 | | Palmitoyl Oligopeptide | |
| 221227-05-0 | | Palmitoyl Tetrapeptide-7 | |
| N/A | Fragrance - | Fragrance (Parfum) | 0.95% |
| | “Silky Skin” - | | |
| | #6110985 | | |
| 112945-52-5 | ChronoSphere | Silica | 1.00% |
| N/A | Opticals Brite | Polyurethane-40 | |
| 4403-90-1 | | Green 5 | |

### Example 6

### Representative Composition of the PFC Cream

TABLE 6
Fifth Representative Composition

| CAS # | Trade Name | INCI Name | Wt. % |
|---|---|---|---|
| N/A | Perfluoro-tert- | Perfluoro-tert- | 45.00% |
| | butylclyclohexane | butylclyclohexane | |
| 7732-18-5 | Deionized Water | Water (Aqua) | 18.40% |
| 76050-42-5 | Carbopol Ultrez-10 | Carbomer | 0.70% |
| 504-63-2 | Zemea | Propanediol | 2.00% |
| 139-33-3 | Dissolvine Na-2-P | Disodium EDTA | 0.16% |
| 91770-40-0 | Avocadin | Persea gratissima | 0.80% |
| | | (Avocado) Oil | |
| | | Unsaponifiables | |
| 68920-03-6 | Shea Butter | Butyrospermum parkii | 1.50% |
| | “Ultra Refined” | (Shea Butter) | |
| 57-11-4 | Stearic Acid | Stearic Acid | 2.50% |
| 3539-43-3 | Amphisol A | Cetyl Phosphate | 2.50% |
| 31566-31-1 | Simulsol 165 | Glyceryl Stearate | 1.50% |
| 9004-99-3 | | PEG-100 Stearate | |
| 63148-62-9 | Dow Corning 200 | Dimethicone | 1.50% |
| | Fluid 350 CST | | |
| 541-02-6 | Volasil 995 | Cyclopentasiloxane | 2.50% |
| 65381-09-1 | Myritol 312 | Caprylic/Capric | 1.00% |
| | | Triglyceride | |
| 541-02-6 | KP-545 | Cyclopentasiloxane | 2.94% |
| N/A | | Acrylates/Dimethicone | |
| | | Copolymer | |
| 8001-21-6 | Soline | Helianthus annuus | 0.10% |
| | | (Sunflower) Seed Oil | |
| | | Unsaponifiables | |
| N/A | Botanol MO | Limnanthes alba | 2.00% |
| | | (Meadowfoam) Seed Oil | |
| 1310-73-2 | Sodium Hydroxide, | Sodium Hydroxide | 2.00% |
| | Pellets, NF | | |
| 122-99-6 | Mikrokill Cos | Phenoxyethanol | 3.00% |
| 1117-86-8 | | Caprylyl Glycol | |
| 104-29-0 | | Chlorphenesin | |
| 68797-35-3 | ARG-DPG | Dipotassium | 0.20% |
| | | Glycyrrhizate | |
| 129499-78-1 | AA2G | Ascorbyl Glucoside | 1.00% |
| 7732-18-5 | Drago-Calm | Water (Aqua) | 0.10% |
| 56-81-5 | 674463 | Glycerin | |
| 84012-26-0 | | Avena sativa (Oat) | |
| | | Kernel Extract | |
| 107-88-0 | Cytobiol | Butylene Glycol | 2.20% |
| 7732-18-5 | Lumin-Eye | Water (Aqua) | |
| 98-92-0 | | Niacinamide | |
| 84625-28-5 | | Fraxinus excelsior | |
| | | Bark Extract | |
| N/A | | Silanetriol | |
| 866-84-2 | | Potassium Citrate | |
| 7732-18-5 | Haloxyl | Water (Aqua) | 1.55% |
| 56-81-5 | | Glycerin | |
| 9005-00-9 | | Steareth-20 | |
| 6066-82-6 | | N-Hydroxysuccinimide | |
| 480-40-0 | | Chrysin | |
| 147732-56-7 | | Palmitoyl Oligopeptide | |
| 221227-05-0 | | Palmitoyl Tetrapeptide-7 | |
| 56-81-5 | Matrixyl 3000 | Glycerin | 2.25% |
| 7732-18-5 | | Water (Aqua) | |
| 107-88-0 | | Butylene Glycol | |
| 76050-42-5 | | Carbomer | |
| 9005-64-5 | | Polysorbate 20 | |
| 147732-56-7 | | Palmitoyl Oligopeptide | |
| 221227-05-0 | | Palmitoyl Tetrapeptide-7 | |
| N/A | Fragrance - | Fragrance (Parfum) | 0.10% |
| | “Silky Skin” - | | |
| | #6110985 | | |
| 112945-52-5 | ChronoSphere | Silica | 2.50% |
| N/A | Opticals Brite | Polyurethane-40 | |
| 4403-90-1 | | Green 5 | |

### Example 7

### Representative Composition of the PFC Cream

TABLE 7
Sixth Representative Composition

| CAS # | Trade Name | INCI Name | Wt. % |
|---|---|---|---|
| N/A | Perfluoro-tert- | Perfluoro-tert- | 30.50% |
| | butylclyclohexane | butylclyclohexane | |
| 7732-18-5 | Deionized Water | Water (Aqua) | 26.30% |
| 76050-42-5 | Carbopol Ultrez-10 | Carbomer | 0.45% |
| 504-63-2 | Zemea | Propanediol | 6.50% |
| 139-33-3 | Dissolvine Na-2-P | Disodium EDTA | 0.20% |
| 91770-40-0 | Avocadin | Persea gratissima | 0.60% |
| | | (Avocado) Oil | |
| | | Unsaponifiables | |
| 68920-03-6 | Shea Butter | Butyrospermum parkii | 4.00% |
| | “Ultra Refined” | (Shea Butter) | |
| 57-11-4 | Stearic Acid | Stearic Acid | 1.05% |
| 3539-43-3 | Amphisol A | Cetyl Phosphate | 4.00% |
| 31566-31-1 | Simulsol 165 | Glyceryl Stearate | 2.80% |
| 9004-99-3 | | PEG-100 Stearate | |
| 63148-62-9 | Dow Corning 200 | Dimethicone | 2.00% |
| | Fluid 350 CST | | |
| 541-02-6 | Volasil 995 | Cyclopentasiloxane | 5.00% |
| 65381-09-1 | Myritol 312 | Caprylic/Capric | 1.20% |
| | | Triglyceride | |
| 541-02-6 | KP-545 | Cyclopentasiloxane | 2.75% |
| N/A | | Acrylates/Dimethicone | |
| | | Copolymer | |
| 8001-21-6 | Soline | Helianthus annuus | 0.04% |
| | | (Sunflower) Seed Oil | |
| | | Unsaponifiables | |
| N/A | Botanol MO | Limnanthes alba | 1.90% |
| | | (Meadowfoam) Seed Oil | |
| 1310-73-2 | Sodium Hydroxide, | Sodium Hydroxide | 0.41% |
| | Pellets, NF | | |
| 122-99-6 | Mikrokill Cos | Phenoxyethanol | 1.50% |
| 1117-86-8 | | Caprylyl Glycol | |
| 104-29-0 | | Chlorphenesin | |
| 68797-35-3 | ARG-DPG | Dipotassium | 1.00% |
| | | Glycyrrhizate | |
| 129499-78-1 | AA2G | Ascorbyl Glucoside | 1.55% |
| 7732-18-5 | Drago-Calm | Water (Aqua) | 0.24% |
| 56-81-5 | 674463 | Glycerin | |
| 84012-26-0 | | Avena sativa (Oat) | |
| | | Kernel Extract | |
| 107-88-0 | Cytobiol | Butylene Glycol | 2.50% |
| 7732-18-5 | Lumin-Eye | Water (Aqua) | |
| 98-92-0 | | Niacinamide | |
| 84625-28-5 | | Fraxinus excelsior | |
| | | Bark Extract | |
| N/A | | Silanetriol | |
| 866-84-2 | | Potassium Citrate | |
| 7732-18-5 | Haloxyl | Water (Aqua) | 1.00% |
| 56-81-5 | | Glycerin | |
| 9005-00-9 | | Steareth-20 | |
| 6066-82-6 | | N-Hydroxysuccinimide | |
| 480-40-0 | | Chrysin | |
| 147732-56-7 | | Palmitoyl Oligopeptide | |
| 221227-05-0 | | Palmitoyl Tetrapeptide-7 | |
| 56-81-5 | Matrixyl 3000 | Glycerin | 1.25% |
| 7732-18-5 | | Water (Aqua) | |
| 107-88-0 | | Butylene Glycol | |
| 76050-42-5 | | Carbomer | |
| 9005-64-5 | | Polysorbate 20 | |
| 147732-56-7 | | Palmitoyl Oligopeptide | |
| 221227-05-0 | | Palmitoyl Tetrapeptide-7 | |
| N/A | Fragrance - | Fragrance (Parfum) | 0.01% |
| | “Silky Skin” - | | |
| | #6110985 | | |
| 112945-52-5 | ChronoSphere | Silica | 1.25% |
| N/A | Opticals Brite | Polyurethane-40 | |
| 4403-90-1 | | Green 5 | |

### Example 8

### Representative Composition of the PFC Cream

TABLE 8
Seventh Representative Composition

| CAS # | Trade Name | INCI Name | Wt. % |
|---|---|---|---|
| N/A | Perfluoro-tert- | Perfluoro-tert- | 20.00% |
| | butylclyclohexane | butylclyclohexane | |
| 7732-18-5 | Deionized Water | Water (Aqua) | 36.20% |
| 76050-42-5 | Carbopol Ultrez-10 | Carbomer | 0.50% |
| 504-63-2 | Zemea | Propanediol | 3.00% |
| 139-33-3 | Dissolvine Na-2-P | Disodium EDTA | 1.00% |
| 91770-40-0 | Avocadin | Persea gratissima | 0.90% |
| | | (Avocado) Oil | |
| | | Unsaponifiables | |
| 68920-03-6 | Shea Butter | Butyrospermum parkii | 1.00% |
| | “Ultra Refined” | (Shea Butter) | |
| 57-11-4 | Stearic Acid | Stearic Acid | 3.00% |
| 3539-43-3 | Amphisol A | Cetyl Phosphate | 2.25% |
| 31566-31-1 | Simulsol 165 | Glyceryl Stearate | 3.00% |
| 9004-99-3 | | PEG-100 Stearate | |
| 63148-62-9 | Dow Corning 200 | Dimethicone | 1.00% |
| | Fluid 350 CST | | |
| 541-02-6 | Volasil 995 | Cyclopentasiloxane | 3.00% |
| 65381-09-1 | Myritol 312 | Caprylic/Capric | 2.00% |
| | | Triglyceride | |
| 541-02-6 | KP-545 | Cyclopentasiloxane | 1.00% |
| N/A | | Acrylates/Dimethicone | |
| | | Copolymer | |
| 8001-21-6 | Soline | Helianthus annuus | 0.50% |
| | | (Sunflower) Seed Oil | |
| | | Unsaponifiables | |
| N/A | Botanol MO | Limnanthes alba | 1.75% |
| | | (Meadowfoam) Seed Oil | |
| 1310-73-2 | Sodium Hydroxide, | Sodium Hydroxide | 0.75% |
| | Pellets, NF | | |
| 122-99-6 | Mikrokill Cos | Phenoxyethanol | 2.10% |
| 1117-86-8 | | Caprylyl Glycol | |
| 104-29-0 | | Chlorphenesin | |
| 68797-35-3 | ARG-DPG | Dipotassium | 0.50% |
| | | Glycyrrhizate | |
| 129499-78-1 | AA2G | Ascorbyl Glucoside | 1.50% |
| 7732-18-5 | Drago-Calm | Water (Aqua) | 0.50% |
| 56-81-5 | 674463 | Glycerin | |
| 84012-26-0 | | Avena sativa (Oat) | |
| | | Kernel Extract | |
| 107-88-0 | Cytobiol | Butylene Glycol | 6.50% |
| 7732-18-5 | Lumin-Eye | Water (Aqua) | |
| 98-92-0 | | Niacinamide | |
| 84625-28-5 | | Fraxinus excelsior | |
| | | Bark Extract | |
| N/A | | Silanetriol | |
| 866-84-2 | | Potassium Citrate | |
| 7732-18-5 | Haloxyl | Water (Aqua) | 2.50% |
| 56-81-5 | | Glycerin | |
| 9005-00-9 | | Steareth-20 | |
| 6066-82-6 | | N-Hydroxysuccinimide | |
| 480-40-0 | | Chrysin | |
| 147732-56-7 | | Palmitoyl Oligopeptide | |
| 221227-05-0 | | Palmitoyl Tetrapeptide-7 | |
| 56-81-5 | Matrixyl 3000 | Glycerin | 2.00% |
| 7732-18-5 | | Water (Aqua) | |
| 107-88-0 | | Butylene Glycol | |
| 76050-42-5 | | Carbomer | |
| 9005-64-5 | | Polysorbate 20 | |
| 147732-56-7 | | Palmitoyl Oligopeptide | |
| 221227-05-0 | | Palmitoyl Tetrapeptide-7 | |
| N/A | Fragrance - | Fragrance (Parfum) | 0.05% |
| | “Silky Skin” - | | |
| | #6110985 | | |
| 112945-52-5 | ChronoSphere | Silica | 3.50% |
| N/A | Opticals Brite | Polyurethane-40 | |
| 4403-90-1 | | Green 5 | |

### Example 9

### Representative Composition of the PFC Cream

TABLE 9
Eighth Representative Composition

| CAS # | Trade Name | INCI Name | Wt. % |
|---|---|---|---|
| N/A | Perfluoro-tert- | Perfluoro-tert- | 35.00% |
| | butylclyclohexane | butylclyclohexane | |
| 7732-18-5 | Deionized Water | Water (Aqua) | 26.30% |
| 76050-42-5 | Carbopol Ultrez-10 | Carbomer | 0.80% |
| 504-63-2 | Zemea | Propanediol | 2.50% |
| 139-33-3 | Dissolvine Na-2-P | Disodium EDTA | 0.08% |
| 91770-40-0 | Avocadin | Persea gratissima | 0.85% |
| | | (Avocado) Oil | |
| | | Unsaponifiables | |
| 68920-03-6 | Shea Butter | Butyrospermum parkii | 3.00% |
| | “Ultra Refined” | (Shea Butter) | |
| 57-11-4 | Stearic Acid | Stearic Acid | 1.90% |
| 3539-43-3 | Amphisol A | Cetyl Phosphate | 1.90% |
| 31566-31-1 | Simulsol 165 | Glyceryl Stearate | 3.25% |
| 9004-99-3 | | PEG-100 Stearate | |
| 63148-62-9 | Dow Corning 200 | Dimethicone | 1.25% |
| | Fluid 350 CST | | |
| 541-02-6 | Volasil 995 | Cyclopentasiloxane | 2.00% |
| 65381-09-1 | Myritol 312 | Caprylic/Capric | 1.75% |
| | | Triglyceride | |
| 541-02-6 | KP-545 | Cyclopentasiloxane | 1.75% |
| N/A | | Acrylates/Dimethicone | |
| | | Copolymer | |
| 8001-21-6 | Soline | Helianthus annuus | 0.25% |
| | | (Sunflower) Seed Oil | |
| | | Unsaponifiables | |
| N/A | Botanol MO | Limnanthes alba | 1.40% |
| | | (Meadowfoam) Seed Oil | |
| 1310-73-2 | Sodium Hydroxide, | Sodium Hydroxide | 0.46% |
| | Pellets, NF | | |
| 122-99-6 | Mikrokill Cos | Phenoxyethanol | 1.30% |
| 1117-86-8 | | Caprylyl Glycol | |
| 104-29-0 | | Chlorphenesin | |
| 68797-35-3 | ARG-DPG | Dipotassium | 0.06% |
| | | Glycyrrhizate | |
| 129499-78-1 | AA2G | Ascorbyl Glucoside | 3.00% |
| 7732-18-5 | Drago-Calm | Water (Aqua) | 0.11% |
| 56-81-5 | 674463 | Glycerin | |
| 84012-26-0 | | Avena sativa (Oat) | |
| | | Kernel Extract | |
| 107-88-0 | Cytobiol | Butylene Glycol | 3.85% |
| 7732-18-5 | Lumin-Eye | Water (Aqua) | |
| 98-92-0 | | Niacinamide | |
| 84625-28-5 | | Fraxinus excelsior | |
| | | Bark Extract | |
| N/A | | Silanetriol | |
| 866-84-2 | | Potassium Citrate | |
| 7732-18-5 | Haloxyl | Water (Aqua) | 2.00% |
| 56-81-5 | | Glycerin | |
| 9005-00-9 | | Steareth-20 | |
| 6066-82-6 | | N-Hydroxysuccinimide | |
| 480-40-0 | | Chrysin | |
| 147732-56-7 | | Palmitoyl Oligopeptide | |
| 221227-05-0 | | Palmitoyl Tetrapeptide-7 | |
| 56-81-5 | Matrixyl 3000 | Glycerin | 3.20% |
| 7732-18-5 | | Water (Aqua) | |
| 107-88-0 | | Butylene Glycol | |
| 76050-42-5 | | Carbomer | |
| 9005-64-5 | | Polysorbate 20 | |
| 147732-56-7 | | Palmitoyl Oligopeptide | |
| 221227-05-0 | | Palmitoyl Tetrapeptide-7 | |
| N/A | Fragrance - | Fragrance (Parfum) | 0.07% |
| | “Silky Skin” - | | |
| | #6110985 | | |
| 112945-52-5 | ChronoSphere | Silica | 1.98% |
| N/A | Opticals Brite | Polyurethane-40 | |
| 4403-90-1 | | Green 5 | |

### Example 10

### Representative Composition the PFC Cream

TABLE 10
Ninth Representative Composition

| CAS # | Trade Name | INCI Name | Wt. % |
|---|---|---|---|
| N/A | Perfluoro-tert- | Perfluoro-tert- | 25.00% |
| | butylclyclohexane | butylclyclohexane | |
| 7732-18-5 | Deionized Water | Water (Aqua) | 34.00% |
| 76050-42-5 | Carbopol Ultrez-10 | Carbomer | 0.10% |
| 504-63-2 | Zemea | Propanediol | 5.00% |
| 139-33-3 | Dissolvine Na-2-P | Disodium EDTA | 0.03% |
| 91770-40-0 | Avocadin | Persea gratissima | 0.25% |
| | | (Avocado) Oil | |
| | | Unsaponifiables | |
| 68920-03-6 | Shea Butter | Butyrospermum parkii | 3.20% |
| | “Ultra Refined” | (Shea Butter) | |
| 57-11-4 | Stearic Acid | Stearic Acid | 1.00% |
| 3539-43-3 | Amphisol A | Cetyl Phosphate | 1.00% |
| 31566-31-1 | Simulsol 165 | Glyceryl Stearate | 4.80% |
| 9004-99-3 | | PEG-100 Stearate | |
| 63148-62-9 | Dow Corning 200 | Dimethicone | 1.75% |
| | Fluid 350 CST | | |
| 541-02-6 | Volasil 995 | Cyclopentasiloxane | 2.00% |
| 65381-09-1 | Myritol 312 | Caprylic/Capric | 3.00% |
| | | Triglyceride | |
| 541-02-6 | KP-545 | Cyclopentasiloxane | 5.00% |
| N/A | | Acrylates/Dimethicone | |
| | | Copolymer | |
| 8001-21-6 | Soline | Helianthus annuus | 0.20% |
| | | (Sunflower) Seed Oil | |
| | | Unsaponifiables | |
| N/A | Botanol MO | Limnanthes alba | 1.40% |
| | | (Meadowfoam) Seed Oil | |
| 1310-73-2 | Sodium Hydroxide, | Sodium Hydroxide | 0.50% |
| | Pellets, NF | | |
| 122-99-6 | Mikrokill Cos | Phenoxyethanol | 2.00% |
| 1117-86-8 | | Caprylyl Glycol | |
| 104-29-0 | | Chlorphenesin | |
| 68797-35-3 | ARG-DPG | Dipotassium | 0.04% |
| | | Glycyrrhizate | |
| 129499-78-1 | AA2G | Ascorbyl Glucoside | 2.00% |
| 7732-18-5 | Drago-Calm | Water (Aqua) | 0.20% |
| 56-81-5 | 674463 | Glycerin | |
| 84012-26-0 | | Avena sativa (Oat) | |
| | | Kernel Extract | |
| 107-88-0 | Cytobiol | Butylene Glycol | 1.50% |
| 7732-18-5 | Lumin-Eye | Water (Aqua) | |
| 98-92-0 | | Niacinamide | |
| 84625-28-5 | | Fraxinus excelsior | |
| | | Bark Extract | |
| N/A | | Silanetriol | |
| 866-84-2 | | Potassium Citrate | |
| 7732-18-5 | Haloxyl | Water (Aqua) | 1.50% |
| 56-81-5 | | Glycerin | |
| 9005-00-9 | | Steareth-20 | |
| 6066-82-6 | | N-Hydroxysuccinimide | |
| 480-40-0 | | Chrysin | |
| 147732-56-7 | | Palmitoyl Oligopeptide | |
| 221227-05-0 | | Palmitoyl Tetrapeptide-7 | |
| 56-81-5 | Matrixyl 3000 | Glycerin | 2.50% |
| 7732-18-5 | | Water (Aqua) | |
| 107-88-0 | | Butylene Glycol | |
| 76050-42-5 | | Carbomer | |
| 9005-64-5 | | Polysorbate 20 | |
| 147732-56-7 | | Palmitoyl Oligopeptide | |
| 221227-05-0 | | Palmitoyl Tetrapeptide-7 | |
| N/A | Fragrance - | Fragrance (Parfum) | 0.03% |
| | “Silky Skin” - | | |
| | #6110985 | | |
| 112945-52-5 | ChronoSphere | Silica | 2.00% |
| N/A | Opticals Brite | Polyurethane-40 | |
| 4403-90-1 | | Green 5 | |

### Example 11

### Representative Composition of the PFC Cream

TABLE 11
Tenth Representative Composition

| CAS # | Trade Name | INCI Name | Wt. % |
|---|---|---|---|
| N/A | Perfluoro-tert- | Perfluoro-tert- | 30.00% |
| | butylclyclohexane | butylclyclohexane | |
| 7732-18-5 | Deionized Water | Water (Aqua) | 27.50% |
| 76050-42-5 | Carbopol Ultrez-10 | Carbomer | 0.20% |
| 504-63-2 | Zemea | Propanediol | 3.50% |
| 139-33-3 | Dissolvine Na-2-P | Disodium EDTA | 0.05% |
| 91770-40-0 | Avocadin | Persea gratissima | 0.75% |
| | | (Avocado) Oil | |
| | | Unsaponifiables | |
| 68920-03-6 | Shea Butter | Butyrospermum parkii | 3.50% |
| | “Ultra Refined” | (Shea Butter) | |
| 57-11-4 | Stearic Acid | Stearic Acid | 2.20% |
| 3539-43-3 | Amphisol A | Cetyl Phosphate | 2.30% |
| 31566-31-1 | Simulsol 165 | Glyceryl Stearate | 4.00% |
| 9004-99-3 | | PEG-100 Stearate | |
| 63148-62-9 | Dow Corning 200 | Dimethicone | 1.30% |
| | Fluid 350 CST | | |
| 541-02-6 | Volasil 995 | Cyclopentasiloxane | 1.10% |
| 65381-09-1 | Myritol 312 | Caprylic/Capric | 3.25% |
| | | Triglyceride | |
| 541-02-6 | KP-545 | Cyclopentasiloxane | 2.00% |
| N/A | | Acrylates/Dimethicone | |
| | | Copolymer | |
| 8001-21-6 | Soline | Helianthus annuus | 0.28% |
| | | (Sunflower) Seed Oil | |
| | | Unsaponifiables | |
| N/A | Botanol MO | Limnanthes alba | 1.60% |
| | | (Meadowfoam) Seed Oil | |
| 1310-73-2 | Sodium Hydroxide, | Sodium Hydroxide | 0.70% |
| | Pellets, NF | | |
| 122-99-6 | Mikrokill Cos | Phenoxyethanol | 1.00% |
| 1117-86-8 | | Caprylyl Glycol | |
| 104-29-0 | | Chlorphenesin | |
| 68797-35-3 | ARG-DPG | Dipotassium | 0.07% |
| | | Glycyrrhizate | |
| 129499-78-1 | AA2G | Ascorbyl Glucoside | 1.76% |
| 7732-18-5 | Drago-Calm | Water (Aqua) | 0.13% |
| 56-81-5 | 674463 | Glycerin | |
| 84012-26-0 | | Avena sativa (Oat) | |
| | | Kernel Extract | |
| 107-88-0 | Cytobiol | Butylene Glycol | 4.00% |
| 7732-18-5 | Lumin-Eye | Water (Aqua) | |
| 98-92-0 | | Niacinamide | |
| 84625-28-5 | | Fraxinus excelsior | |
| | | Bark Extract | |
| N/A | | Silanetriol | |
| 866-84-2 | | Potassium Citrate | |
| 7732-18-5 | Haloxyl | Water (Aqua) | 2.75% |
| 56-81-5 | | Glycerin | |
| 9005-00-9 | | Steareth-20 | |
| 6066-82-6 | | N-Hydroxysuccinimide | |
| 480-40-0 | | Chrysin | |
| 147732-56-7 | | Palmitoyl Oligopeptide | |
| 221227-05-0 | | Palmitoyl Tetrapeptide-7 | |
| 56-81-5 | Matrixyl 3000 | Glycerin | 3.00% |
| 7732-18-5 | | Water (Aqua) | |
| 107-88-0 | | Butylene Glycol | |
| 76050-42-5 | | Carbomer | |
| 9005-64-5 | | Polysorbate 20 | |
| 147732-56-7 | | Palmitoyl Oligopeptide | |
| 221227-05-0 | | Palmitoyl Tetrapeptide-7 | |
| N/A | Fragrance - | Fragrance (Parfum) | 0.06% |
| | “Silky Skin” - | | |
| | #6110985 | | |
| 112945-52-5 | ChronoSphere | Silica | 3.00% |
| N/A | Opticals Brite | Polyurethane-40 | |
| 4403-90-1 | | Green 5 | |

### Example 12

### Manufacturing the Perfluorocarbon Cream

A perfluorocarbon cream was manufactured in 5 phases according to the Table 12 below:

TABLE 12

| Item No. | Trade Name | Processing Container |
|---|---|---|
| Phase A | | |
| 1 | Deionized Water | Main Processing Tank |
| 2 | Carbopol Ultrez-10 | Main Processing Tank |
| 3 | Zemea | Main Processing Tank |
| 4 | Dissolvine Na-2-P | Main Processing Tank |
| Phase B | | |
| 5 | Avocadin | Auxiliary Tank |
| 6 | Shea Butter “Ultra | Auxiliary Tank |
| | Refined” | |
| 7 | Stearic Acid | Auxiliary Tank |
| 8 | Amphisol A | Auxiliary Tank |
| 9 | Simulsol 165 | Auxiliary Tank |
| 10 | Dow Corning 200 | Auxiliary Tank |
| | Fluid 350 CST | |
| 11 | Volasil 995 | Auxiliary Tank |
| 12 | Myritol 312 | Auxiliary Tank |
| 13 | KP-545 | Auxiliary Tank |
| 14 | Soline | Auxiliary Tank |
| 15 | Botanol MO | Auxiliary Tank |
| Phase C | | |
| 16 | Deionized Water | Auxiliary Tank |
| 17 | Sodium Hydroxide, | Auxiliary Tank |
| | Pellets, NF | |
| Phase D | | |
| 18 | Mikrokill Cos | Main Processing Tank |
| 19 | ARG-DPG | Main Processing Tank |
| 20 | AA2G | Main Processing Tank |
| 21 | Drago-Calm 674463 | Main Processing Tank |
| 22 | Cytobiol Lumin-Eye | Main Processing Tank |
| 23 | Haloxyl | Main Processing Tank |
| 24 | Matrixyl 3000 | Main Processing Tank |
| 25 | perfluoro(tert- | Main Processing Tank |
| | butylcyclohexane) | |
| 26 | Fragrance - “silky | Main Processing Tank |
| | Skin” - #6110985 | |
| Phase E | | |
| 27 | Deionized Water | Auxiliary Tank |
| 28 | ChronoSphere | Auxiliary Tank |
| | Opticals Brite | |

The following manufacturing procedures were followed:
1. Phase A:
   a. Add Item No. 1 (Deionized water) into the main processing tank.
   b. Start high speed mixing.
   c. Add Item No. 2.
   d. Mix until completely dispersed.
   e. Heat to 80° C.-85° C.
   f. Add Item Nos. 3 and 4.
   g. Mix until uniform.
   h. Maintain temperature.
2. Phase B:
   a. In a separate tank, add Item Nos. 5-15.
   b. Heat to 80° C.-85° C.
   c. Mix until all the solids are completely dissolved.
   d. Add Phase B to Phase A.
   e. Mix until uniform.
3. Phase C:
   a. In a separate container, add Item Nos. 16 and 17.
   b. Mix until all the solids are completely dissolved.
   c. Add Phase C to the main tank.
   d. Mix for 30 minutes until uniform.
   e. Cool to 40° C.
4. Phase D:
   a. At 40° C., add Item Nos. 18-26, mixing well after each addition.
5. Phase E:
   a. In a separate container, add Item Nos. 27 and 28.
   b. Mix until completely homogeneous.
   c. Add homogenous mixture to the main tank.
   d. Mix until uniform. Homogenize if necessary.
6. Continue mixing and cooling to 35° C. Mix for 20 minutes or until uniform.

Results

The resulting product had characteristics as shown in Table 13:

TABLE 13
Product Characteristics

| Color: | Light blue to light green |
|---|---|
| Appearance | Opaque, viscous cream |
| pH at 25° C. | 6.00-7.00 |
| Viscosity at 25° C. (RVT: | 10,000-20,000 cps |
| Spindle 5 @ 10 rpm) | |
| Specific gravity at 25 25° C. | 1.14-1.18 |
| Total Aerobic Plate Count: | Less than 100 cfu/g |
| Yeast & Mold: | Less than 100 cft/g |
| P. Aeruginosa: | Absent |
| S. Aureus: | Absent |
| Percent Solids at 130° C. | 19.80%-22.00% |

### Example 13

### Perfluorocarbon Cream For Cosmetic Applications

### Example 13A

A perfluorocarbon cream composition as described herein is topically administered to the periocular skin a subject in need thereof.

Topical administration of the PFC cream is effective to improve the overall appearance of the subject's periocular skin by reducing the appearance of or the severity of fine lines, wrinkles, puffiness, dark (under-eye) circles, bags and/or dark blemishes in the subjects' skin.

### Example 13B

A perfluorocarbon cream composition as described herein is topically administered to the periocular skin of a subject.

Topical administration of the PFC cream is effective to increases oxygen delivery to the periocular skin of the subject. In addition, the perfluorocarbon cream is well tolerated and has no toxicity.

### Example 14

### An 8-Week Clinical Study of the Effects of an Oxygen-Rich Perfluorocarbon Composition on Skin Appearance

The periodic topical application of an oxygen-rich composition comprising perfluoro(tert-butylcyclohexane) to subjects' skin improved the skin's overall appearance. The PFC composition is formulated as follows:

| Component | Wt % |
|---|---|
| perfluoro(tert-butylcyclohexane) | 86.00 |
| Water | 10.25 |
| Pluronic ® L35 (Poloxamer 105) | 2.45 |
| Gluconolactone, Sodium Benzoate, Calcium Gluconate | 1.00 |
| Pluronic ® F-108 (Poloxamer 388) | 0.30 |

A significant portion of the study subjects showed at least one grade improvement on the Fitzpatrick Wrinkle Assessment Scale (FWAS) beginning at the four-week time point, as determined by the investigators. There was a significant difference between 4, 5, 6, 7 and 8-week FWAS scores and those from baseline. At the conclusion of the study conducted with women ages 39-63 with mild-to-moderate facial wrinkles, 80.65% of subjects exhibited at least one-grade improvement on the FWAS (P<0.0001) with 38.71% showing at least a two grade improvement.

There was a statistically significant increase in favorable responses for the subject Global Aesthetic Improvement Scale (GAIS) score beginning with the five-week time point and continuing through the eight-week time point. This increase suggests that the subjects perceived improvement of their skin's overall appearance, or at least the maintenance of their skin's overall appearance. Investigator GAIS scores also showed significant improvement beginning at the three-week time point and continuing through the study. At the conclusion of the study 97% of the subjects experienced at least one grade of improvement compared to baseline according to the investigator's GAIS (P<0.0001). 84% of the subjects have at least one grade of improvement on their self-perceived GAIS score (P<0.0001).

Finally, the Skin Replica data suggests that the PFC composition had a mild smoothing effect on the subjects' fine lines and wrinkles.

Study Results

Fitzpatrick Wrinkle Assessment Scale (FWAS)

The investigator assessed the degree of facial wrinkling and elastosis at all visits. The investigator was asked to perform a live facial assessment of the subject using the FWAS, a 10-point categorical scale corresponding to 0 (None, no wrinkling or elastosis), 1-3 (Mild, fine wrinkles and fine textual changes with subtly accentuated skin lines), 4-6 (Moderate, fine to moderate depth wrinkles, moderate number of lines, and distinct popular elastosis), and 7-10 (Severe, fine to deep wrinkles, numerous lines with or without redundant skin folds, and multipapular and confluent elastosis). In order to be enrolled in the clinical study, subjects were required to have a FWAS grade of Mild to Moderate corresponding to a FWAS score of 1-6.

Table 14 below shows the number of subjects with each score at Visits V1-V9. Significant improvement in FWAS score as compared to baseline was observed at the 4-week (Visit 5) time point and continued through the duration of the study.

TABLE 14
FWAS number of total assessments for each grade for
all subjects completing the indicated visit

| | Score | | | | | | | | |
|---|---|---|---|---|---|---|---|---|---|
| Grade | V1 | V2 | V3 | V4 | V5 | V6 | V7 | V8 | V9 |
| None | 0 | 0 | 0 | 0 | 0 | 0 | 0 | 0 | 0 |
| (0) | | | | | | | | | |
| Mild | 10 | 11 | 10 | 11 | 13 | 13 | 14 | 16 | 20 |
| 1 | 0 | 0 | 0 | 0 | 0 | 1 | 1 | 1 | 1 |
| 2 | 0 | 0 | 2 | 4 | 8 | 8 | 8 | 8 | 7 |
| 3 | 10 | 11 | 8 | 7 | 5 | 4 | 5 | 7 | 12 |
| Mod- | 26 | 23 | 22 | 21 | 17 | 16 | 15 | 14 | 11 |
| erate | | | | | | | | | |
| 4 | 8 | 9 | 7 | 7 | 10 | 10 | 14 | 12 | 9 |
| 5 | 7 | 4 | 6 | 5 | 6 | 6 | 1 | 2 | 2 |
| 6 | 11 | 10 | 9 | 9 | 1 | 0 | 0 | 0 | 0 |
| Se- | 0 | 0 | 0 | 0 | 0 | 0 | 0 | 0 | 0 |
| vere | | | | | | | | | |
| 7 | 0 | 0 | 0 | 0 | 0 | 0 | 0 | 0 | 0 |
| 8 | 0 | 0 | 0 | 0 | 0 | 0 | 0 | 0 | 0 |
| 9 | 0 | 0 | 0 | 0 | 0 | 0 | 0 | 0 | 0 |
| P | | NS | NS | NS | 0.002 | <0.001 | <0.001 | <0.001 | <0.001 |

Table 14 shows a significant difference between baseline FWAS scores and FWAS scores from Visits 5, 6, 7, 8 and 9 with the n umber of lower scores increasing with length of time the subjects were applying the PFC composition.

Table 15 below shows the percentage of total subject completing the visit with each FWAS score.

TABLE 15
FWAS percentage of total assessments for each grade
for all subjects completing the indicated visit
Score

| Grade | V1 | V2 | V3 | V4 | V5 | V6 | V7 | V8 | V9 |
|---|---|---|---|---|---|---|---|---|---|
| None (0) | 0.00% | 0.00% | 0.00% | 0.00% | 0.00% | 0.00% | 0.00% | 0.00% | 0.00% |
| Mild | 27.78% | 32.35% | 31.25% | 34.38% | 43.33% | 44.83% | 48.28% | 53.33% | 64.52% |
| 1 | 0.00% | 0.00% | 0.00% | 0.00% | 0.00% | 3.45% | 3.45% | 3.33% | 3.23% |
| 2 | 0.00% | 0.00% | 6.25% | 12.50% | 26.67% | 27.59% | 27.59% | 26.67% | 22.58% |
| 3 | 27.78% | 32.35% | 25.00% | 21.88% | 16.67% | 13.79% | 17.24% | 23.33% | 38.71% |
| Moderate | 72.22 | 67.65 | 68.75 | 65.63 | 56.67 | 55.17 | 51.72 | 46.67 | 35.48 |
| 4 | 22.22 | 26.47 | 21.88 | 21.88 | 33.33 | 34.48 | 48.28 | 40.00 | 29.03 |
| 5 | 19.44% | 11.76% | 18.75% | 15.63% | 20.00% | 20.69% | 3.45% | 6.67% | 6.45% |
| 6 | 30.56% | 29.41% | 28.13% | 28.13% | 3.33% | 0.00% | 0.00% | 0.00% | 0.00% |
| Severe | 0.00% | 0.00% | 0.00% | 0.00% | 0.00% | 0.00% | 0.00% | 0.00% | 0.00% |
| 7 | 0.00% | 0.00% | 0.00% | 0.00% | 0.00% | 0.00% | 0.00% | 0.00% | 0.00% |
| 8 | 0.00% | 0.00% | 0.00% | 0.00% | 0.00% | 0.00% | 0.00% | 0.00% | 0.00% |
| 9 | 0.00% | 0.00% | 0.00% | 0.00% | 0.00% | 0.00% | 0.00% | 0.00% | 0.00% |

Table 16 below shows the change in FWAS score as compared to baseline score. The change in FWAS score was calculated by subtracting the subject's indicated visit FWAS score from their baseline FWAS score. A negative grade change corresponds to an improvement in FWAS. Significant improvement in FWAS score change as compared to visit 2 was observed at the 4-week (Visit 5) time point and continued through the duration of the study. This data suggests that there was a statistically significant increase in the number of subjects experiencing improvement in the degree of wrinkling and elastosis according to FWAS.

TABLE 16
Change in FWAS score from baseline for all subjects
completing the indicated visit

| Change | | | | | | | | |
|---|---|---|---|---|---|---|---|---|
| in Score | V2 | V3 | V4 | V5 | V6 | V7 | V8 | V9 |
| −3 | 0 | 0 | 0 | 0 | 0 | 2 | 2 | 5 |
| −2 | 0 | 1 | 1 | 7 | 8 | 10 | 11 | 7 |
| −1 | 0 | 20 | 18 | 8 | 6 | 5 | 5 | 6 |
| 0 | 31 | 20 | 18 | 8 | 6 | 2 | 5 | 6 |
| +1 | 0 | 4 | 3 | 1 | 1 | 2 | 0 | 0 |
| P | | NS | NS | <0.001 | <0.001 | <0.001 | <0.001 | <0.001 |

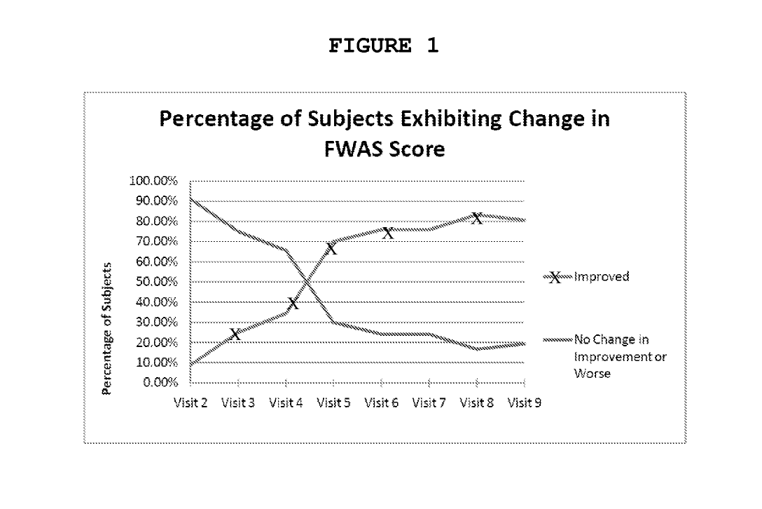

Table 17 below shows the results for the categorization of response. A negative grade corresponds to at least 1 grade improvement on the FWAS and a positive change or no change in grade corresponds to no improvement or a worsened condition. FIG. 1 illustrates the progression of improvement as observed throughout the study.

TABLE 17
Change in FWAS where scores were evaluated as either
improved vs. no improvement or worsened

| | | | | | | | | |
|---|---|---|---|---|---|---|---|---|
| Change in Score | V2 | V3 | V4 | V5 | V6 | V7 | V8 | V9 |

Improved

| | | | | | | | | |
|---|---|---|---|---|---|---|---|---|
| N | 3 | 8 | 11 | 21 | 22 | 22 | 25 | 25 |
| % | 8.82 | 25.00 | 34.38 | 70.00 | 75.86 | 75.86 | 83.33 | 80.65 |

Not Improved or Worse

| | | | | | | | | |
|---|---|---|---|---|---|---|---|---|
| N | 31 | 24 | 21 | 9 | 7 | 7 | 5 | 6 |
| % | 91.18 | 75.00 | 65.63 | 30.00 | 24.14 | 24.14 | 16.67 | 19.35 |

Global Aesthetic Improvement Scale (GAIS)

The data was assessed for both the subject and the investigator impressions of how the treatment had an effect on the overall appearance of facial skin using the GAIS, a 5-point categorical scale consisting of the responses worse, no change, improved, much improved, or very much improved. The subject and investigator were asked to complete a GAIS at visits 2-9 by comparing a photograph from the current visit to a photograph from the subject's baseline visit.

Table 18 below shows the number of responses for each effect (worse, no change, improved, much improved, and very much improved). The data suggests a significant difference in the number of responses at visits 6-9 as compared to Visit 2, the time point of initial GAIS.

TABLE 18
Subject GAIS number of total responses for all
subjects completing the indicated visit

| Grade | V2 | V3 | V4 | V5 | V6 | V7 | V8 | V9 |
|---|---|---|---|---|---|---|---|---|
| Very Much | 1 | 1 | 1 | 1 | 1 | 1 | 1 | 3 |
| Improved | | | | | | | | |
| Much | 0 | 1 | 0 | 2 | 4 | 4 | 3 | 3 |
| Improved | | | | | | | | |
| Improved | 10 | 8 | 17 | 13 | 11 | 12 | 18 | 20 |
| No Change | 20 | 22 | 14 | 14 | 13 | 12 | 8 | 5 |
| Worse | 3 | 0 | 0 | 0 | 0 | 0 | 0 | 0 |
| P | | NS | NS | 0.055 | 0.024 | 0.015 | 0.002 | <0.001 |

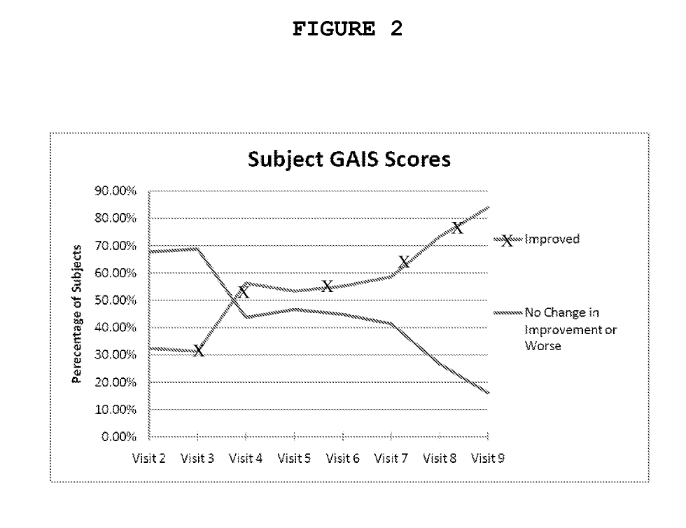

Table 19 below shows the results where the responses were categorized into either ‘No Change in Improvement or Worse’ or ‘Improvement’. To be categorized as ‘No Change in Improvement or Worse’, the subject must have responded ‘No Change’ or “Worse’. In contrast, to be categorized as ‘Improved’, a subject was required to have responded ‘Improved’ or ‘Much Improved’ or ‘Very Much Improved’. FIG. 2 illustrates the progression of subjects' GAIS scores throughout the study.

TABLE 19
Subject GAIS number of responses where respondents
state either no or less benefit vs. more benefit

| | | | | | | | | |
|---|---|---|---|---|---|---|---|---|
| Change in Score | V2 | V3 | V4 | V5 | V6 | V7 | V8 | V9 |

Improved

| | | | | | | | | |
|---|---|---|---|---|---|---|---|---|
| N | 11 | 10 | 18 | 16 | 16 | 17 | 22 | 26 |
| % | 32.35 | 31.35 | 56.25 | 53.33 | 55.17 | 58.62 | 73.33 | 83.87 |

Not Improved or Worse

| | | | | | | | | |
|---|---|---|---|---|---|---|---|---|
| N | 23 | 22 | 14 | 14 | 13 | 12 | 8 | 5 |
| % | 67.65 | 68.75 | 43.75 | 46.67 | 44.83 | 41.38 | 26.67 | 16.13 |

Table 20 below shows the investigator's GAIS score from the investigator's consideration of the photograph of the subject's appearance. The data suggests that significant improvement was noticeable beginning at the 3-week (Visit 4) time point and continuing through the duration of the study with progressively more significance and number of subjects exhibiting improvement.

The difference in the initial time point in which significance was observed between the two investigator assessments (FWAS and GAIS) may be attributed to a subtle improvement in appearance that could be perceived using the GAIS, but not a large enough improvement to constitute a change of FWAS score.

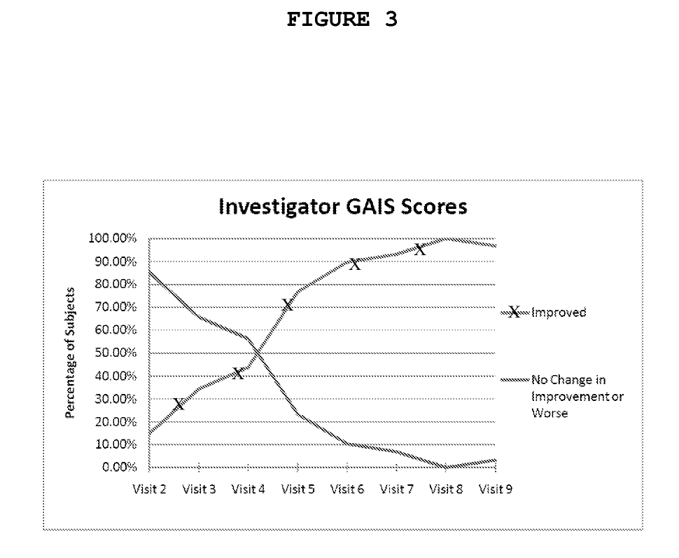

The investigator's GAIS responses were also categorized as ‘No Change in Improvement or Worse’ and “Improved’ (Table 21). FIG. 3 illustrates the progression of investigator GAIS scores throughout the study.

TABLE 20
Subject GAIS number of total responses for all
subject completing the indicated visit

| Grade | V2 | V3 | V4 | V5 | V6 | V7 | V8 | V9 |
|---|---|---|---|---|---|---|---|---|
| Very Much Improved | 0 | 1 | 0 | 0 | 0 | 0 | 0 | 1 |
| Much Improved | 0 | 0 | 0 | 1 | 4 | 7 | 5 | 4 |
| Improved | 5 | 10 | 14 | 22 | 22 | 20 | 25 | 25 |
| No Change | 29 | 21 | 18 | 7 | 3 | 2 | 0 | 1 |
| Worse | 0 | 0 | 0 | 0 | 0 | 0 | 0 | 0 |
| P | | NS | 0.009 | <0.001 | <0.001 | <0.001 | <0.001 | <0.001 |

TABLE 21
Investigator GAIS number of responses stating either
no or less benefit vs. more benefit

| | | | | | | | | |
|---|---|---|---|---|---|---|---|---|
| Change in Score | V2 | V3 | V4 | V5 | V6 | V7 | V8 | V9 |

Improved

| | | | | | | | | |
|---|---|---|---|---|---|---|---|---|
| N | 5 | 11 | 14 | 23 | 26 | 27 | 30 | 30 |
| % | 14.71 | 34.38 | 43.75 | 76.67 | 89.66 | 93.10 | 100.0 | 96.77 |

Not Improved or Worse

| | | | | | | | | |
|---|---|---|---|---|---|---|---|---|
| N | 29 | 21 | 18 | 7 | 3 | 2 | 0 | 1 |
| % | 85.29 | 65.63 | 56.25 | 23.33 | 10.34 | 6.90 | 0.00 | 3.23 |

Skin Replica Data

Skin Replica silicon profilometry was performed at baseline, 4-weeks (Visit 5) and 8-weeks (Visit 9). The major and minor lines are measured by 8 parameters separated into two groups of 4. Group A parameters define the luminance along a set of 10 equal length parallel lines (or passes) running across the replica and are parallel to the direction of lighting. The variations within the luminance are treated as indications of the skin's roughness, representing major lines and are analyzed using surface roughness statistics.

Group A parameters are:
Rz—the average maximum difference in luminance value for 5 equal length segments in each of the 10 lines that are traversing the sample.
Ra—the average deviation of the luminance curve about the mean luminance for the same 10 lines measured in Rz.

These ‘R’ measures are reported in the units of brightness or Gray levels ranging from 0-255:
FSpace—the distance between markers placed on the lines at luminance changes indicative of fine lines.
FNum—the number of markers per millimeter placed on the lines at luminance changes indicative of fine lines.

Group B parameters represent minor lines assess the replica image area by dividing it into 10 equal width bands (or sub areas). The shadow-like features are detected in each of the bands according to their luminance values as compared to those less than the detection threshold.

Four parameters are determined from detected features:
1. Spacing—the mean distance in millimeters between adjacent detected features (i.e. spacing between midpoints of adjacent shadowy features).
2. Breadth—the average breadth in millimeters of the detected feature and is proportional to the depth of the wrinkle producing the shadow.
3. Shadows—the percent of the sampled replica area with luminance values less than the detected threshold. This is the relative area of shadows cast by the wrinkles and fine lines within the replica.
4. NumWr—the total number of features detected in the 10 bands or sub areas used to calculate spacing and breadth.

To interpret these parameters within the major and minor lines associated with the Crow's Feet area of the face; the following assessment pointers were used:
The measurement's Rz and Ra tend to increase with increasing roughness.
As lines and creases of the face disappear due to a particular treatment regimen, FSpace increases and FNum decreases. Spacing can decrease with conversion of deep wrinkles to fine wrinkles (moisturization) and increases with disappearance of wrinkles.
Breadth decreases as wrinkles become shallow, but is not sensitive to the number or length of wrinkles.
Shadows tend to decrease with smoothing of the skin, and is sensitive to both the length and depth of wrinkles
NumWr decreases with smoothing of the skin (fewer visible features).

The Skin Replica results are presented in Tables 22 and 23. The N values are nominal values. For the Spacing parameter, the actual N used to calculate the statistics was smaller than the nominal value due to insufficient detected line/wrinkle features in one of the replica images to permit calculation.

Changes from baseline were calculated by subtracting each subject's baseline values from the appropriate subsequent values. The mean changes were tested for significance using the one sample t-test against a value of zero. The P value associated with the t statistic was tabulated with the appropriate means, standard deviations and t-values.

Table 22 shows mean (SD) values for replica data for parameters that assess major lines around the area of the eye where Crow's Feet typically develop. Changes from baseline showed a non-significant trend for one parameter, Breadth, at 8-weeks (Visit 9) (P=0.0660). The decrease in Breadth was in the direction of smoother texture.

Table 23 details mean (SD) for values obtained for the minor lines that are associated with the Crow's Feet area around the eye. Changes from baseline for two parameters were significant at 8-weeks (Visit 9). For both Breadth (P=0.009) and Shadows (P=0.05), the changes were in the direction of smoother fine line texture. The results suggest a mild smoothing effect on the fine lines and wrinkles, as 2 of the smoothness parameters measured were affected.

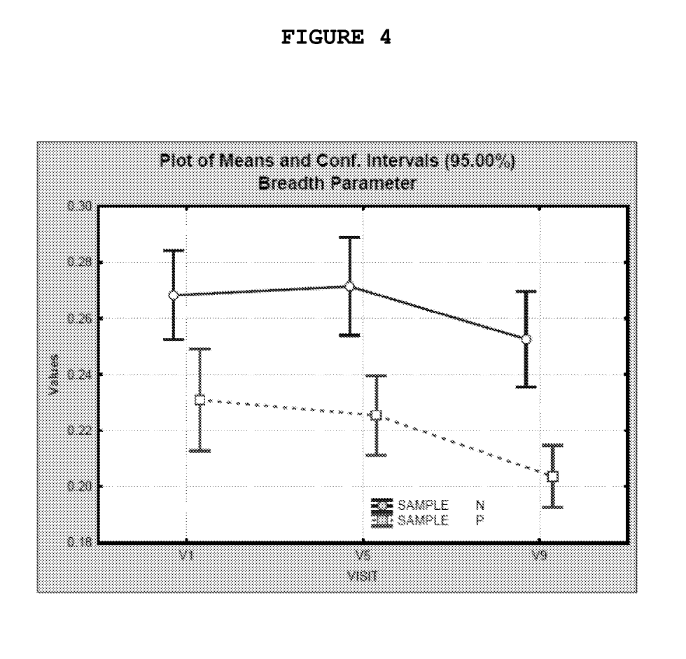

FIG. 4 represents a plot of means and confidence intervals (95.00%) for the Breadth parameter, which experienced significant change towards a smooth appearance at 8-weeks (Visit 9).

TABLE 22
Skin Replica mean values and difference between means (SD)
for major lines for all subjects completing the study

| | | | V5 | | | | V9 | | |
|---|---|---|---|---|---|---|---|---|---|
| | V1 | | (N = 28) | | | | (N = 31) | | |
| | (N = 31) | | Mean | | | | Mean | | |
| Parameter | Mean | Mean | Dif. | t-value | P | Mean | Dif. | t-value | P |
| Rz | 150.0 | 148.8 | 1.9 | 0.2903 | NS | 150.6 | 0.5 | 0.0884 | NS |
| | (30.5) | (30.7) | (35.2) | | | (29.6) | (33.7) | | |
| Ra | 31.8 | 31.8 | 0.9 | 0.5526 | NS | 31.7 | 0.0 | −0.0250 | NS |
| | (7.5) | (8.2) | (8.8) | | | (7.9) | (7.9) | | |
| FNUM | 0.426 | 0.423 | 0.01 | 0.2428 | NS | 0.435 | 0.01 | 0.4001 | NS |
| | (0.111) | (0.113) | (0.12) | | | (0.112) | (0.12) | | |
| IDL | 5.81 | 5.60 | −0.04 | −0.0983 | NS | 6.20 | 0.39 | 1.0828 | NS |
| | (1.60) | (1.58) | (1.92) | | | (1.61) | (1.98) | | |
| Spacing | 1.273 | 1.300 | −0.022 | −0.1851 | NS | 1.295 | 0.022 | 0.1998 | NS |
| | (0.542) | (0.400) | (0.642) | | | (0.499) | (0.610) | | |
| Breadth | 0.268 | 0.271 | 0.006 | 0.7845 | NS | 0.252 | −0.016 | −1.9077 | 0.0660 |
| | (0.043) | (0.045) | (0.042) | | | (0.046) | (0.046) | | |
| Shadows | 9.2 | 9.1 | 0.5 | 0.5068 | NS | 8.5 | −0.7 | −0.7766 | NS |
| | (5.0) | (4.5) | (5.6) | | | (4.9) | (5.2) | | |
| NumWr | 106.4 | 104.2 | 3.2 | 0.3943 | NS | 107.5 | 1.1 | 0.1375 | NS |
| | (40.8) | (33.4) | (43.1) | | | (42.1) | (44.4) | | |

TABLE 23
Skin Replica mean values and difference between means (SD)
for minor lines for all subjects completing the study

| | | | V5 | | | | V9 | | |
|---|---|---|---|---|---|---|---|---|---|
| | V1 | | (N = 28) | | | | (N = 31) | | |
| | (N = 31) | | Mean | | | | Mean | | |
| Parameter | Mean | Mean | Dif. | t-value | P | Mean | Dif. | t-value | P |
| Rz | 131.6 | 125.3 | −6.3 | −0.9074 | NS | 129.0 | −2.7 | −0.4048 | NS |
| | (33.2) | (22.7) | (36.5) | | | (24.4) | (37.0) | | |
| Ra | 28.0 | 26.7 | −1.3 | −0.7533 | NS | 26.9 | −1.1 | −0.7035 | NS |
| | (8.1) | (5.9) | (9.0) | | | (5.7) | (8.6) | | |
| FNUM | 0.439 | 0.425 | −0.02 | −0.7522 | NS | 0.422 | −0.02 | −0.7289 | NS |
| | (0.104) | (0.095) | (0.11) | | | (0.107) | (0.12) | | |
| IDL | 5.02 | 4.67 | −0.38 | −1.0859 | NS | 5.38 | 0.36 | 0.9968 | NS |
| | (1.62) | (1.12) | (1.85) | | | (1.49) | (2.02) | | |
| Spacing | 1.927 | 1.899 | −0.055 | −0.3264 | NS | 1.922 | −0.032 | −0.1490 | NS |
| | (0.939) | (0.606) | (0.889) | | | (0.867) | (1.184) | | |
| Breadth | 0.231 | 0.225 | −0.007 | −0.6268 | NS | 0.204 | −0.027 | −2.7951 | 0.0090 |
| | (0.050) | (0.037) | (0.058) | | | (0.030) | (0.054) | | |
| Shadows | 5.0 | 4.1 | −0.9 | −0.8646 | NS | 3.2 | −1.8 | −2.0426 | 0.0500 |
| | (4.4) | (2.9) | (5.2) | | | (2.5) | (4.8) | | |
| NumWr | 66.8 | 59.4 | −6.9 | −0.7597 | NS | 50.6 | −16.2 | −1.6603 | NS |
| | (43.8) | (37.0) | (48.3) | | | (35.9) | (54.4) | | |

Statistical Methods

Statistical significance was defined as a P value of 0.05 or less, with a 95% confidence interval. A non-significant trend was defined as a P value of greater than 005 and less than 0.10.

For FWAS and GAIS, the number of responses for each category was recorded and analyzed using a paired T-test. The FWAS data was then reassessed to evaluate the difference as compared to baseline analyzed using a paired T-test. FWAS and GAIS data were then reassessed to evaluate differences in respondents reporting ‘no change or worse’ with those who reported ‘improved’ or ‘much improved’ or ‘very much improved’.

The skin replica data was analyzed by assessment of the change from baseline as calculated by subtracting each subject's baseline values from the appropriate subsequent visit values. Mean changes form baseline were tested for significance using the one sample t-test against a value of zero.

Discussion

Fitzpatrick Wrinkle Assessment Scale (FWAS) results suggest that were significantly more subjects exhibiting improvement in fine lines and skin elastosis as the study progressed. Significance was first observed at the four-week time point and continued to increase in significance through eight weeks. Change in FWAS results suggest that subjects begin to see improvements at four weeks with the majority of subjects exhibiting some improvement by eight weeks.

Results observed for the Global Aesthetic Improvement Scale (GAIS) suggest that there were significantly more subjects who reported that their appearance was improved the longer they were applying the PFC composition. Significance was first observed with the five-week time point, although three-week data (P=0.064) and four week data (P=0.055) suggest non-significant trends. The number of subjects responding ‘improved’, ‘much improved’ or ‘very much improved’ continued to increase through the different time points increasing in significance at each subsequent visit.

Investigator GAIS data similarly suggests significantly more subjects demonstrating improvement in overall appearance the longer they were applying the PFC composition. Significance was demonstrated at the three-week time point and continued throughout the study. It is postulated that the investigator GAIS scores were significant at an earlier time point than subject GAIS scores because the investigators are more experienced in observing facial characteristics such as fine lines, elastosis, skin tone, and overall complexion.

While investigator GAIS data was significant at three weeks, FWAS data was not significant until four-weeks. It is hypothesized that this difference is due to earlier changes in skin tone and overall complexion that are able to be documented through the GAIS, but are not part of the specific categories of the FWAS.

FWAS, subject GAIS and investigator GAIS results suggest the majority of subjects show signs of improvement at the four-week time point. While more subjects exhibited signs in aesthetic improvement at the study time points progressed, the change in FWAS results suggests that there is not an increasing amount of improvement the longer a subject uses the PFC composition. That is to say that there appears not to be an exponential increase in skin improvement after six weeks, but rather maintenance of the improved results.

Skin Replica data results show a significant smoothing effect of minor fine lines around the Crow's Feet area of the eye for those subjects applying the PFC composition over the 8-week duration of the study. These results were significant for the breadth and shadows parameters suggesting a mild smoothing effect. This might suggest that the treatment had subtle effects on the collagen matrix of the skin and might indicate that the PFC composition acted at the cellular level, providing subtle softening effects for the fine lines around the eye.

The results of the study suggest that the application of the PFC composition (twice daily) improves the appearance of fine lines and overall texture of the skin after a period of 4-6 weeks.

### REFERENCES

1. U.S. Pat. No. 4,490,351, issued Dec. 25, 1984 to Leland Clark Jr.
2. U.S. Pat. No. 4,857,304, issued Aug. 15, 1989 to Ishiwatari, et al.
3. U.S. Pat. No. 5,643,601, issued Jul. 1, 1997 to Gross, et al.

1. A method of delivering oxygen to a periocular skin of a subject comprising topically administering to a facial area consisting of the periocular skin of the subject a composition comprising a perfluorocarbon effective to deliver oxygen to the periocular skin.

2. A method of improving the appearance of a periocular skin of a subject comprising topically administering to a facial area consisting of the periocular skin of the subject a composition comprising a perfluorocarbon effective to improve the appearance of the periocular skin.

3. The method of claim 1, wherein the molecular formula of the perfluorocarbon consists fluorine atoms and 9-12 carbon atoms.

4. The method of claim 3, wherein the perfluorocarbon is perfluoro(tert-butylcyclohexane).

5. The method of claim 1, wherein the composition is in the form of a cream.

6. The method of claim 1, wherein the composition is administered periodically.

7. The method of claim 6, wherein the composition is administered twice daily.

8. The method of claim 6, wherein the administration is for a period of greater than 3 weeks.

9. The method of claims 8, wherein the administration is for a period of 8 weeks or more.

10. The method of any one of claim 1, wherein the subject's Fitzpatrick Wrinkle Assessment Scale score is decreased.

11. The method of claim 10, wherein the subject's Fitzpatrick Wrinkle Assessment Scale score is decreased by at least 1 point.

12. The method of claim 10, wherein the subject's Fitzpatrick Wrinkle Assessment Scale score is decreased by at least 2 points.

13. The method of claim 1, wherein the subject's Global Aesthetic Improvement Scale score is improved.

14. The method of claim 1, wherein the improvement in appearance is the reduction of the severity of fine lines, wrinkles, skin elastosis, puffiness, dark circles, under-eye circles, bags and/or dark blemishes.

15. A perfluorocarbon cream composition comprising 1) a perfluorocarbon, 2) ascorbyl glucoside, 3) a first mixture comprising butylene glycol, water, niacinamide, Fraxinus excelsior bark extract, silanetriol, and potassium citrate, 4) a second mixture comprising water, glycerin, steareth-20, N-hydroxysuccinimide, chrysin, palmitoyl oligopeptide and palmitoyl tetrapeptide-7 and 5) a third mixture comprising glycerin, water, butylene glycol, carbomer, polysorbate 20, palmitoyl oligopeptide, and palmitoyl tetrapeptide-7.

16-32. (canceled)

33. The method of claim 1, comprising topically administering to the subject the perfluorocarbon cream composition of claim 15.

34. The method of claim 2, comprising topically administering to the subject the perfluorocarbon cream composition of claim 15.
